(19) United States
(12) Patent Application Publication
(10) Pub. No.: US 2023/0255516 A1
(43) Pub. Date: Aug. 17, 2023

(54) System and Method for Detection of Biomolecules in Tissues, Organs, and Extracellular Fluid

(71) Applicant: THE REGENTS OF THE UNIVERSITY OF CALIFORNIA, Oakland, CA (US)

(72) Inventors: Jeffrey Laurence ARDELL, Encino, CA (US); Corey SMITH, University Heights, OH (US); Kalyanam SHIVKUMAR, Los Angeles, CA (US)

(21) Appl. No.: 18/002,995

(22) PCT Filed: Jun. 25, 2021
(86) PCT No.: PCT/US2021/039104

§ 371 Date: Dec. 22, 2022

Related U.S. Application Data
(60) Provisional application No. 63/044,083, filed on Jun. 25, 2020.

(51) Int. Cl. A61B 5/145; A61B 5/263

(52) U.S. Cl. CPC A61B 5/14503; A61B 5/263; A61B 2562/0214

(57) ABSTRACT

The present invention provides a device and methods of use related to the use of electrodes to continuously detect the presence and abundance of various biochemical compounds of interest with high spatial and temporal resolution, comprising the steps of inserting one or more electrodes in one or more locations selected from the group consisting of a tissue, an organ, a neural structure, a lymphatic vessel, a lymphatic node, an extravascular fluid compartment, and a peripheral blood vessel; applying a voltage scan to the electrode; an detecting a current indicative of the presence and abundance of the compound.

### STATEMENT REGARDING FEDERALLY SPONSORED RESEARCH OR DEVELOPMENT

This invention was made with government support under Grant Number EB025138, awarded by the National Institutes of Health. The government has certain rights in the invention.

### BACKGROUND OF THE INVENTION

Catecholamines and other neurotransmitters are produced by central neurons, peripheral autonomic sympathetic neurons and neuroendocrine chromaffin cells of the adrenal gland and serve a variety of functions in normal physiology and pathophysiology. When released in the central and peripheral nervous systems they can function as neuromediators/neuromodulators and when released in the blood circulation, they can function as hormones. Currently, there is no means by which to directly measure the concentration of catecholamine or other neuromediators/neuromodulators in near real-time in the heart or vascular compartment under normal conditions or in response to stressors. The current state of the art in monitoring cardiac autonomic function or dysfunction uses blood tests or tissue biopsy, which are less accurate and carry a higher risk of infection or tissue scarring.

Thus, there is a need in the art for a system and method for precise detection and monitoring of neurotransmitters in the heart to evaluate cardiac function or dysfunction. There is also a need in the art for a system and method for precise detection of proteins, protein fragments and biomarkers to aid in evaluation of other disease states such as cancer, endocrine dysfunction, inflammation and other pathological conditions. The present invention satisfies this unmet need.

### SUMMARY OF THE INVENTION

In one aspect the present method provides a method for detecting a biochemical compound comprising the steps of: inserting one or more electrodes in one or more locations selected from the group consisting of: a tissue, an organ, a neural structure, a lymphatic vessel, a lymphatic node, an extravascular fluid compartment, and a peripheral blood vessel; applying a voltage scan to the electrode; and detecting a current indicative of the presence and abundance of the compound.

In one embodiment, the one or more electrodes are placed into the myocardium of a heart. In one embodiment, the one or more electrodes are inserted via epicardial or vascular access. In one embodiment, the compound is at least one catecholamine selected from the group consisting of norepinephrine and epinephrine.

In one embodiment, at least one electrode is an electrode selected from the group consisting of: wire electrodes, microwire electrodes, needle electrodes, plunge electrodes, penetrating electrodes, patch electrodes, single shank electrodes, 2D shank electrodes, 3D shank electrodes, and multi-electrode arrays.

In one embodiment, the voltage scan is a fast scanning cyclic voltammetry (FSCV) voltage scan. In one embodiment, the FSCV voltage scan comprises a waveform selected from the group consisting of: a sawtooth pattern or sinusoidal pattern.

In one embodiment, the method comprises detecting the oxidation current of the compound. In one embodiment, the method comprises constructing a voltammogram from the detected current, thereby identifying the signal diagnostic for the compound of interest. In one embodiment, the method comprises quantifying the abundance of the compound by plotting the peak oxidation current on a calibration curve.

In one embodiment, the organ is a heart, and the one or more electrodes are placed in one or more locations selected from the group consisting of: a coronary sinus of the heart, a great vein of the heart, vena cava, left ventricle, aorta, right ventricle, right atria, left atria, pulmonary veins, pulmonary artery, stellate ganglia, dorsal root ganglia, epicardial fat pad, and pericardial fat pad.

In one embodiment, the presence and abundance of the biochemical compound is assessed in response to one or more cardiac stressors. In one embodiment, a plurality of electrodes are placed at a plurality of locations within and around a heart to assess regional differences in the abundance of the biochemical compound.

In one embodiment, the presence and abundance of the biochemical compound is assessed in response to one or more cardio-pulmonary stressors. In one embodiment, a plurality of electrodes are placed at a plurality of locations within and around a heart and lung to assess regional differences in the abundance of the biochemical compound.

In one aspect, the present invention provides a method for detecting a biochemical compound comprising the steps of: inserting one or more electrodes in one or more locations selected from the group consisting of: a tissue, an organ, a neural structure, a lymphatic vessel, a lymphatic node, an extravascular fluid compartment, and a peripheral blood vessel, wherein at least one electrode comprises a receptor molecule that specifically binds the biochemical compound; and detecting a change in the capacitance of the electrode thereby indicating the presence of the biochemical compound.

In one embodiment, the biochemical compound is a protein or peptide that specifically binds to the receptor molecule. In one embodiment, the level of the compound is detected in at least one ganglia selected from the group consisting of intrathoracic ganglia, stellate ganglia, autonomic ganglia, nodose ganglia, dorsal root ganglia and petrosal ganglia. In one embodiment, the one or more electrodes are placed in a peripheral artery or peripheral vein.

In one embodiment, the one or more electrodes are placed into a tissue or organ via direct access. In one embodiment, the one or more electrodes are placed into a tissue or organ via transcutaneous access. In one embodiment, the one or more electrodes are placed into a tissue or organ via vascular access

In one aspect, the present invention provides a biochemical compound detection device, comprising: a controller, comprising a voltage clamp circuit and signal acquisition and amplification device; a reference electrode communicatively connected to the controller; and a one or more measurement electrodes communicatively connected to the controller; wherein the controller is configured to measure a reference potential across the reference and ground electrodes and voltage clamp of the one or more measurement electrodes relative to the reference potential with a defined sawtooth, sinusoidal or step command potential, and to measure the current passing through the one or more measurement electrodes over time; and wherein one or more measurement electrodes are configured to measure the presence and concentration of one or more biochemical compounds.

In one embodiment, at least one measurement electrode comprises a receptor molecule that specifically binds to a biochemical compound. In one embodiment, the device further comprises a semi-permeable membrane applied to a portion of an electrode selected from the group consisting of the reference electrode, the measurement electrode, and the ground electrode. In one embodiment, at least one of the electrodes selected from the group consisting of the measurement electrode and the reference electrode are made of platinum.

In one embodiment, the reference electrode and one or more measurement electrodes each has a conductive substrate layer deposited on the electrode surface suitable for attachment/binding of IgG antibodies, IgG binding fragments (Fab), single-domain antibody fragments, and peptide binding domain fragments. In one embodiment, the conductive substrate layer is polydopamine. In one embodiment, the controller further comprises a voltage clamp, configured to maintain a substantially constant voltage across two or more electrodes.

In one aspect, the present invention provides a biochemical compound detection device, comprising: a controller, comprising a voltage clamp amplifier; a reference electrode communicatively connected to the controller; a ground electrode communicatively connected to the controller; and one or more sensing electrodes communicatively connected to the controller, each of the one or more sensing electrodes being voltage clamped to a template of positive and negative voltage steps; wherein the controller is configured to measure an electric potential across the reference electrode, the ground electrode, and to apply a command potential relative to the reference potential through a voltage clamp to one or more sensing electrodes, and to measure the current passing through one or more sensing electrodes over time; and wherein one or more sensing electrodes are configured to measure the presence and concentration of one or more biochemical compounds.

In one embodiment, sensitivity of the device is reset by applying a negative potential pulse configured to expel target molecules from capture agents on each of the one or more sensing electrodes, readying the capture agents for a subsequent binding of target molecules for further detection events.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 depicts a schematic of an exemplary use of voltammetry for diagnostic and therapeutic use.

FIG. 2 depicts a schematic of an exemplary embodiment of a method of the invention as described herein.

FIG. 3 depicts an exemplary graphic user interface (GUI) for the control of parameters for fast scanning cyclic voltammetry (FSCV) and capacitive immunosensor (CI) acquisition. The interface was written in the IGOR Pro environment (WaveMetrics, Inc.).

FIG. 4 depicts the voltage clamp circuit for the initial FSCV as described herein.

FIG. 5A through FIG. 5D depict exemplary elements of FSCV. FIG. 5A depicts an exemplary voltage scan delivered to an electrode. FIG. 5B depicts exemplary raw FSCV currents to continuously repeated scans as displayed in FIG. 5A. Current versus time is recorded through a carbon electrode. A two second current is shown. FIG. 5C depicts a voltammogram demonstrating the current at baseline and in the presence of epinephrine. FIG. 5D depicts the oxidation current of epinephrine, obtained by subtracting out the background current.

FIG. 6A and FIG. 6B depict exemplary FSCV recordings for the detection of norepinephrine (NE) (FIG. 6A) and epinephrine (Epi) (FIG. 6B) at known concentrations. The depicted results indicate that Norepinephrine has a unique current versus voltage profile from that of Epinephrine, indicating the signal from these two catecholamines is separable and distinct.

FIG. 7 depicts exemplary calibration curves for quantifying the concentration of norepinephrine (left) and epinephrine (right) from a measured current in picoamperes (pA) using FSCV. These examples are representative for carbon electrodes.

FIG. 8A through FIG. 8D depict the results of electrode design and characterization for in vivo application in a beating heart. Acquisition and analysis software was developed in-house to drive a custom designed 4 channel voltage-clamp amplifier. Perfluoroalkoxy (PFA)-insulated platinum wires, 127 μM in diameter and 30 cm in length, were used as flexible FSCV electrodes to accommodate movement of the heart (FIG. 8A). A sawtooth command waveform (FIG. 8B) drove the recorded voltammograms (FIG. 8C, FIG. 8D). Recordings were performed in bicarbonate-buffered saline (BBS) to mimic the interstitial conditions of the myocardium. A sample voltammogram of an electrode in BBS displays a hysteresis at a scan rate of 12 V/s from −0.5 V to 1.2 V (FIG. 8D).

FIG. 9A through FIG. 9C depict the results of in vitro assessments of electrode sensitivity and stability. Electrodes were superfused with BBS supplemented with increasing concentrations of NE (0 to 2 μM) in a laminar flow chamber. Currents were measured at the peak NE oxidation potential and are presented as a function of time (FIG. 9A). Peak currents at the NE oxidation potential were measured and plotted (FIG. 9B). After recording peak currents at the NE oxidation potential by repeating addition of the given concentrations of NE over 6-hours, the electrodes were found to be stable over this period (FIG. 9C)

FIG. 10 illustrates a recording condition for FSCV and CI in vivo. Sensors are deployed to various sites of the heart and are attached to the amplifier head stages (upper right, blue and silver boxes).

FIG. 11A through FIG. 11D depict the results of in vivo assessments of electrode sensitivity and stability. A platinum electrode was inserted into the left ventricle (LV) mid-myocardium with aid of a hypodermic needle (FIG. 11A). Interstitial NE levels were evaluated at baseline and in response to bilateral stellate ganglion stimulation. Data are presented as a kymograph (FIG. 11B) with Y-axis columns representing the up-stroke of the sawtooth command potential, and time represented on the X axis. Current magnitude is color-coded. The black horizontal line represents the peak oxidation potential for NE. There is emergence of a signal during stellate ganglia stimulation, which persists somewhat after stimulation, indicating increased NE at the electrode tip. Example voltammograms (current vs. command potential) are provided in FIG. 11C. Also provided in FIG. 11C is the NE level measured (FIG. 11B) and calibrated against a standard curve (from FIG. 9B). In simultaneous hemodynamic measurements, complementary increases in heart rate (HR), LV peak systolic pressure (LVSP) and LV developed pressure (dP/dt) were recorded during peak stellate ganglia stimulation (FIG. 11D).

FIG. 12A through FIG. 12C depict the results of experiments measuring interstitial NE levels across multiple regions of the myocardium utilizing 4 independent acquisition channels to provide a gross spatial map of NE levels across the left ventricle in response to acute occlusion of the left anterior descending coronary artery (LAD, 180 s duration). Electrodes were placed caudal to the site of vessel occlusion (indicated by black arrow) within basal regions of the LV whose circulation remains intact (indicated by green and black dots, FIG. 12A). Another set of electrodes were placed apical to the site of occlusion where circulation is blocked (indicated by red and blue dots). FSCV was performed spanning a time-frame 60 s prior to, during occlusion, and into the reperfusion phase. FIG. 12B provides the kymographs for each channel (indicated by the colored dot to the left of each kymograph). As in FIG. 11B, black horizontal lines indicate the peak potential for NE oxidation. Line profiles for current magnitude were pulled as a function of time from the kymographs, calibrated against the standard curve, and plotted (FIG. 12C).

FIG. 13A through FIG. 13C depict the results of experiments measuring NE under varied autonomic and cardiac interventions correlated to hemodynamic responses measured simultaneously. NE release was evaluated during transient occlusions of the descending aorta (AO; FIG. 13A; an increase in afterload) or inferior vena cava (IVC; FIG. 13B; a decrease in preload) and induction of premature ventricular contractions via programmed pacing (PVC; FIG. 13C). Hemodynamic responses were measured, with peak values shown during each stress respectively (right column).

FIG. 14A depicts a schematic of an exemplary capacitive immunosensor. Antibodies are covalently bound to the tip of an electrode, platinum or carbon in this embodiment. The mis-matched conductivity at the electrode interface with the interstitial fluid or blood forms a Helmholz layer characterized by a capacitance at the electrode surface. Mismatched epitopes (black open dots) for the bound antibody do not significantly alter the capacitance. However, binding of the appropriate, specific epitope (red diamonds) to the antibody alters the charge at the electrode tip and results in an increase in the capacitance at the electrode tip. FIG. 14B; Electrode capacitance is measured by step-wise command potential (V_{c}, black line) in the electrode. Two positive step potentials are applied as a control for non-specific oxidative current not related to ligand binding, with equal amplitudes expected in each for a purely capacitive response. Measured current (black current [pA] curves below red stepped command potential lines [mV]) represents the charging function of the electrode with the time constant T measuring the resistance and capacitance (RC) of the system and the amplitude measuring the combined charge required to charge the capacitor and the ohmic current passed through the electrode. FIG. 14C; Capacitance, a function of ligand/biomarker binding is calculated from T and current amplitude.

FIG. 15A depicts the results of CI peptide calibration. (Upper panel) Measured CI signal was obtained for known concentrations of appropriate, matched epitope, enkephalin (Enk, red bars) and report a signal proportional to Enk concentration. Measurements were conducted in TRIS buffered saline and the “TRIS” point represents no Enk in the bath. Parallel negative control measurements with the GAPDH negative control, mismatched epitope probe showed no signal. (open bars). FIG. 15B; a standard calibration curve is constructed for CI signal against Enk concentration.

FIG. 16 depicts the results of CI neurotransmitter detection from ex vivo perfusate. A schematic of the recording protocol is provided in the upper left, with specific and non-specific ligand presented to the CI probe. Enkephalin release was elicited from a hemisected rat adrenal gland. Release was evoked by direct electrical stimulation of the innervating nerve. Measured signal are quantified for Enk and a negative control probe manufactured to detect GAPDH, a non-secretory protein not expected to be released from the adrenal under nerve stimulation. The elevated signal amplitude for the Enk electrode indicates but not GAPDH indicates specific detection of released Enk. FIG. 16, right panel depicts the results of CI Enk calibration. Enk-specific current measured from the adrenal gland is calibrated against the standard curve from FIG. 15 and shows that the concentration of Enk measured from the rat adrenal under nerve stimulation is 132 pM, thus demonstrating the calibration strategy for capacitive immunoprobe detection of peptide transmitters.

FIG. 17 depicts a schematic of resetting the CI sensor to provide a time-resolved signal. The positive step potentials pictured in FIG. 14B are simplified to a single step and are highlighted in red shading. Between each round of capacitance measure, the electrode is clamped at a negative potential (blue shading) to repel the ligand/biomarker from the antibodies. Protein ligand/biomarkers are negatively charged and the negative electric field established by the negative command potential results in electrostatic repulsion, resetting the antibody for a subsequent round of detection.

FIG. 18 depicts the results of time-resolved measure of NPY under ventricular pacing. The detection strategy described in FIG. 17, positive detection pulse, negative reset pulse, to allow for continuous, time-resolved CI measurements. NPY and actin electrode signals were measured under ventricular pacing, a strong autonomic stressor. In this case, actin represents a non-secreted negative control to indicate specificity of the experimental NPY signal. In response to this stressor, a rapid onset, dynamic signal was measured for the NPY probe, but no signal in the immediately adjacent negative control actin probe. The decrease in NPY signal after cessation of the pacing stimulus demonstrates the efficacy of the reset potential approach to provide a time-resolved capacitive signal.

FIG. 19 depicts the results of time-resolved measure of NPY under stellate ganglion stimulation. The detection strategy is again provided in iconographic form above the data plot. Elevated cardiac function was evoked by direct bilateral stimulation of the stellate ganglion (“BSG”). Stellate ganglia are the source for the sympathetic efferent nerves that innervate the heart and release norepinephrine (FIG. 11B) and NPY under strong autonomic stressors. Direct electrical stimulation of the BSG results in a robust, dynamic signal in the NPY probe, no signal in the negative control actin probe. In another negative control with no bound antibody (ØmAb), no signal was detected.

### DETAILED DESCRIPTION

The present invention provides a system, device, and method for detecting biomolecules in the heart to assess and monitor cardiac function or dysfunction. For example, in certain aspects, the invention relates to the detection of neurotransmitters, including, but not limited to catecholamines, such as epinephrine and norepinephrine. In some aspects, the invention relates to the detection of proteins, protein fragments and biomarkers. For example, in certain embodiments, the invention relates to the detection of neurotransmitters and/or proteins, protein fragments and biomarkers that are released by one or more tissues, cells or by the autonomic nervous system. In certain embodiments, the method relates to the detection of a cardiopulmonary event by detecting and monitoring the presence and/or abundance of neurotransmitters and/or proteins, protein fragments and biomarkers in the heart, lungs/vasculature.

Catecholamines are produced and released by components of the sympathetic autonomic nervous system and serve a variety of functions in the heart under normal physiological and pathophysiological conditions. For example, when released in the central and peripheral nervous systems, catecholamines function as neuromediators/neuromodulators, and when released in the blood circulation, catecholamines function as hormones. The ability to detect expression and concentration of such compounds offers insight into the function or dysfunction of the heart, lungs or intrathoracic autonomic nervous system. The present invention allows for the measurement of neurotransmitters and proteins, protein fragments and biomarkers with high temporal and spatial resolution. The presently described device, system, and method can be used to monitor cardiac and cardiopulmonary autonomic function or dysfunction by measuring and monitoring the presence, abundance, and location of neurotransmitters and proteins in the heart, lungs and vascular supply to both organs.

The ability to measure such compounds in response to stimuli in the heart provides great insight into normal and abnormal function of the heart and lungs and the role that compounds such as catecholamines play in pathophysiology. The present invention provides a device and methods for detecting catecholamines, proteins and protein fragments in addition to other neuromodulators and hormones in order to better determine proper function of effector organs. The ability to detect expression and concentration of such compounds can offer insight into proper function of target organs of such compounds, including the heart, lungs, their vasculature and other organ systems.

The ability to measure regional differences in catecholamines in addition to proteins, protein fragments and biomarkers provides greater insights into normal and abnormal function of the neural-heart/lung interface that can be predictive of adverse outcomes, including potential for arrhythmias, heart failure and respiratory dysfunction. The ability to measure regional differences in catecholamines, proteins, protein fragments and biomarkers provides a methodology to rapidly assess efficacy to therapeutic interventions. The ability to measure regional differences in the vascular compartment for catecholamines, proteins, protein fragments and biomarkers provides greater insight into relevant biomarkers indicative of susceptibility to cardiac and cardiopulmonary pathology and the progression of the cardiovascular and cardiopulmonary disease process.

Current strategies for detecting catecholamines in the cardiac setting include microdialysis of the interstitial fluid, followed by off-line detection by high performance liquid chromatography and electrochemical detection. These approaches have a limited temporal resolution of minutes, an analytic time requirement of minutes to hours and are accomplished in a diagnostic lab setting. The process described herein has a temporal resolution on the milliseconds time scale, an analytic time requirement of minutes to near real-time and can be accomplished at the bedside. Moreover, application of the process described herein may be accomplished through a minimally invasive catheter deployment, a characteristic not available to the current methodologies.

In a similar manner, current technologies for detecting and quantifying the presence of proteins, protein fragments and biomarkers typically includes microdialysis of the interstitial fluid, followed by off-line analysis by mass spectrometry, ELISA or HPLC. This approach presents similar technological challenges as those outlined for catecholamine determination, and are similarly limited in determination of spatial distribution and temporal dynamics.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or 0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### DESCRIPTION

The present invention provides a system, device, and method for detecting biomolecules (e.g. proteins, signaling peptides/neuropeptides) in the peripheral tissues/organs, extravascular and vascular fluid compartments and fluids derived from these spaces to assess and monitor biological function or dysfunction. For example, in certain aspects, the invention relates to the detection of neurotransmitters, including but not limited to signaling peptides and amino acids released by nerves within peripheral tissues/organs. In some aspects, the invention relates to the detection of proteins within peripheral tissues/organs. In certain embodiments, the method relates to the detection of biomolecules in vascular space; these molecules being neurotransmitters, neuromodulators or hormones. Access to vascular space allows for trans-organ determination of molecular biomarker or neurotransmitter determination.

The process described herein has a temporal resolution on the milliseconds time scale, an analytic time requirement of minutes to near real-time and can be accomplished at the bedside. The process described herein can provide continuous or sequential biomolecular detection over time frames from seconds to hours to days. Moreover, application of the process described herein may be accomplished through a minimally invasive catheter deployment, a characteristic not available to the current methodologies.

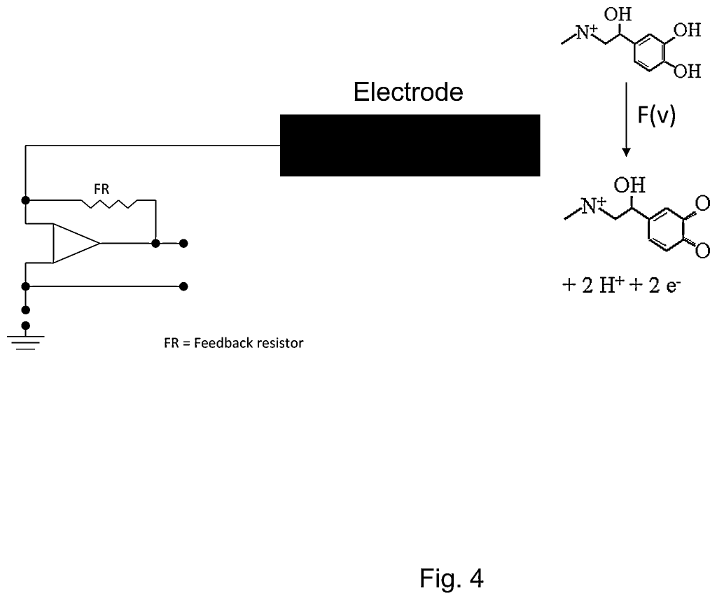

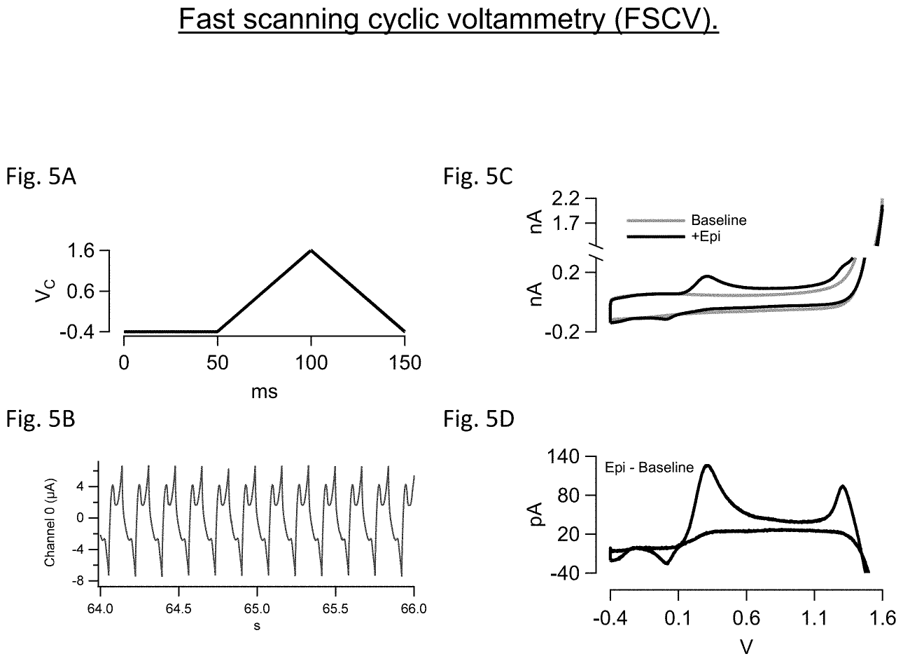

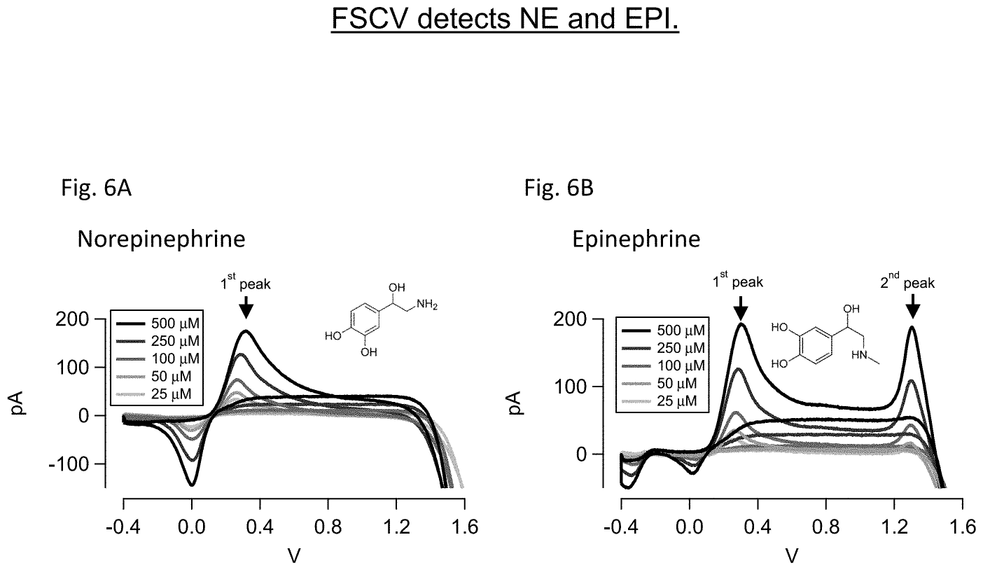

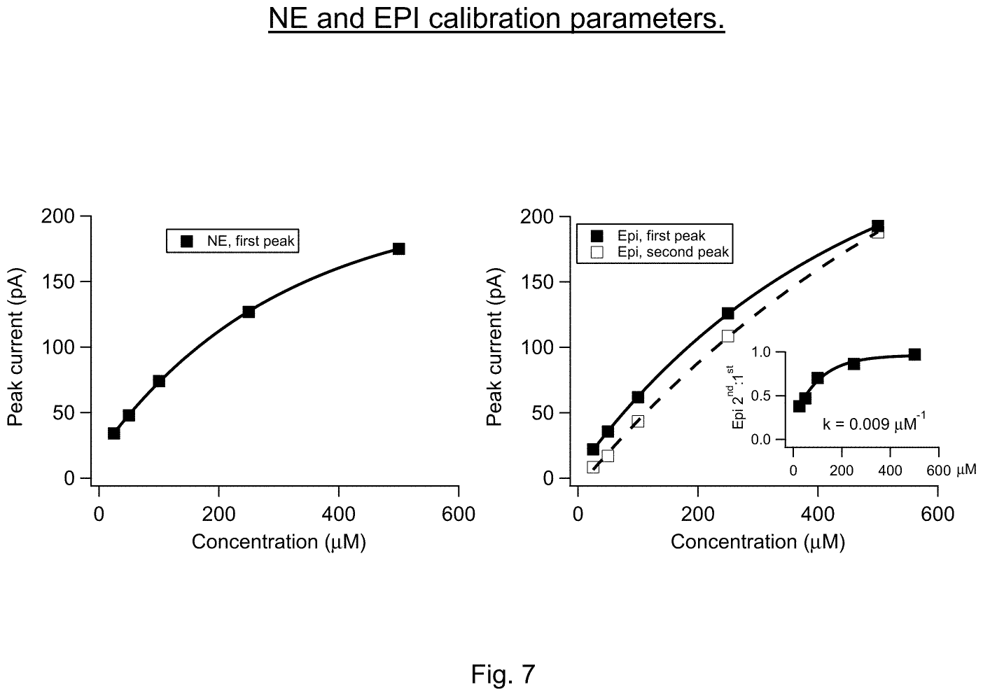

In one aspect, the invention relates to the use of voltammetry to measure the presence and abundance of one or more biomolecules. In some embodiments, the one or more biomolecules includes neurotransmitters, including but not limited to epinephrine and norepinephrine. In a specific embodiment, the invention relates to the use of fast scanning cyclic voltammetry (FSCV), which relates to a technique where the voltage of an implanted electrode is quickly and cyclically increased and then decreased, typically in a triangular or sinusoidal wave pattern. The charge imparted to the electrode sensor zone at the tip generates an electric field, which causes oxidation and reduction reactions of compounds in the vicinity of the electrode tip. The reactions, in turn, induce a measurable current in the electrode through a voltage clamp circuit, for example a voltage clamp circuit as depicted in FIG. 4. Subtraction of the background current from the total current measured produces a voltage versus current plot (i.e. a voltammogram) of the current induced by the oxidation-reduction reactions as depicted in FIG. 5C through FIG. 5D. For example, the characteristic voltammogram produced by the oxidation and reduction of norepinephrine at the electrode tip sensor zone is shown in FIG. 6A, while the characteristic voltammogram produced by the oxidation and reduction of epinephrine at the electrode tip is shown in FIG. 6B. The amplitude of the current at the characteristic peak is correlated with the concentration of the compound present at the vicinity of the electrode tip sensor zone. Higher concentrations of compounds result in more oxidation and reduction reactions, which in turn induce a higher total current as shown in FIG. 6A through FIG. 7. However, the present invention is not limited to the use of FSCV, but rather encompasses the use of any type of voltammetry that induces current from the oxidation and/or reduction of biochemical species in the vicinity of the electrode tip. Other exemplary forms of voltammetry include, but are not limited to, potential step voltammetry, linear sweep voltammetry cyclic voltammetry, square wave voltammetry, staircase voltammetry, anodic or cathodic stripping voltammetry, adsorptive stripping voltammetry, alternating current voltammetry, rotated electrode voltammetry, normal or differential pulse voltammetry, chronoamperometry, and chronocoulometry.

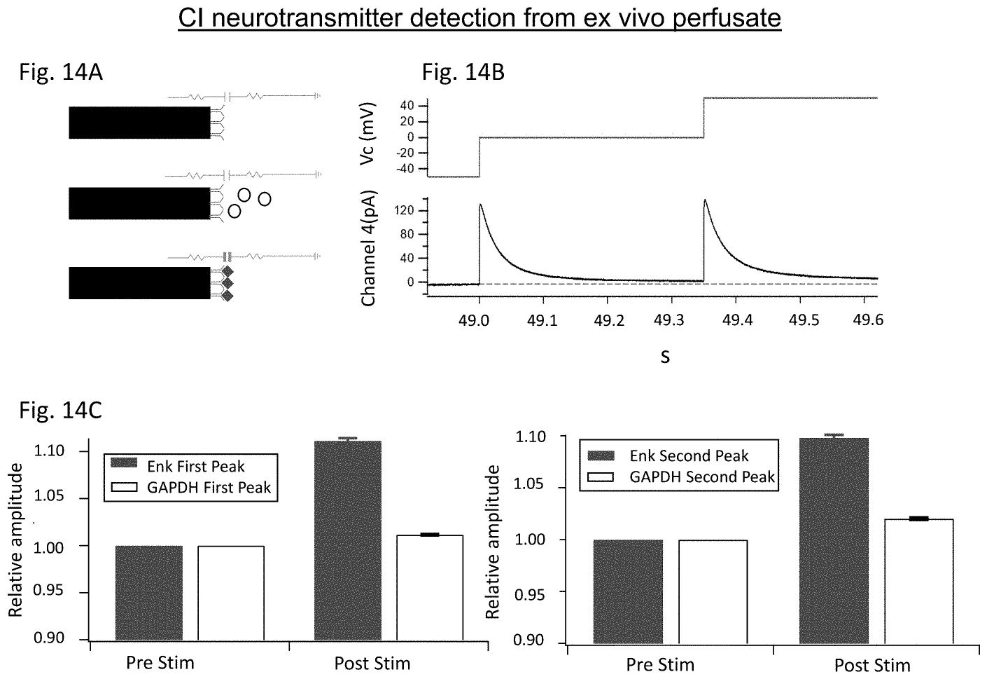

In one embodiment, the invention relates to the use of capacitive immunosensors to detect the presence and abundance of a biochemical compound, such as a protein, peptide, nucleic acid, hormone, or the like in the tissue/organ, extravascular or vascular fluid space or in fluids derived from one or more of these sites. For example, in certain embodiments, the capacitive immunosensors comprise an electrode functionalized with a capture agent, such as an antibody, antibody-fragment, or probe, that specifically binds the biochemical compound. Binding of the compound to the capture agent results in a change in the capacitance of the electrode by displacing water with a static, charged moiety. Thus, a detected change in capacitance is indicative of the presence and abundance of the biochemical compound of interest (FIG. 14A through FIG. 14C).

The present invention provides a device for detecting the presence and abundance of one or more biochemical compounds, including, but not limited to, neurotransmitters, such as epinephrine and norepinephrine, proteins, peptides, nucleic acids, and the like. In one embodiment, the device comprises one or more electrodes configured for implantation into the heart of a subject. The one or more electrodes may comprise any suitable electrode suitable for delivering and measuring a potential. For example, the electrode may comprise a conducting metal, including but not limited to alloys such as indium tin oxide, conductive carbon, or noble metals such as gold, silver, palladium or platinum. Suitable electrodes include, but are not limited to, needle electrodes, plunge electrodes, penetrating electrodes, patch electrodes, single shank electrodes, 2D shank electrodes, 3D shank electrodes, multi-electrode arrays, wire electrodes, microwire electrodes, or the like. In certain embodiments, the device comprises a microelectrode array comprising a plurality of electrode tips suitable for implantation into the target tissue or suitable for placement within the vascular space.

In certain embodiments, the one or more electrodes comprise a wire, microwire, or collection of wires or microwires. In certain embodiments, the electrode comprises a wire electrode having a diameter in the range of about 1 μm to about 5 mm. In one embodiment, the electrode comprises a wire electrode having a diameter in the range of about 10 μm to about 1 mm. In one embodiment, the electrode comprises a wire electrode having a diameter in the range of about 50 μm to about 100 μm. In one embodiment, the electrode comprises a wire electrode having a diameter of about 75 μm. The wire electrode may have any suitable length necessary for implantation into a tissue or region of interest. In certain embodiments the electrode has a length in the range of about 1 mm-500 cm. In certain embodiments the electrode has a length in the range of about 10 mm-100 cm. In certain embodiments the electrode has a length in the range of about 1 cm-50 cm.

In certain embodiments, the electrodes comprise an outer insulation layer. In certain embodiments, the insulation layer comprises a perfluoroalkoxy Teflon (PFA) layer. Other suitable materials of the insulation layer include, but are not limited to glass, a glass coating, silicone, parylene or other suitable material known in the art. In certain embodiment, the insulation layer provides for resistance against thermal or chemical degradation of the electrode. In certain embodiment, the insulation layer provides to restriction of the sensing element(s) to specific part(s) of the wire.

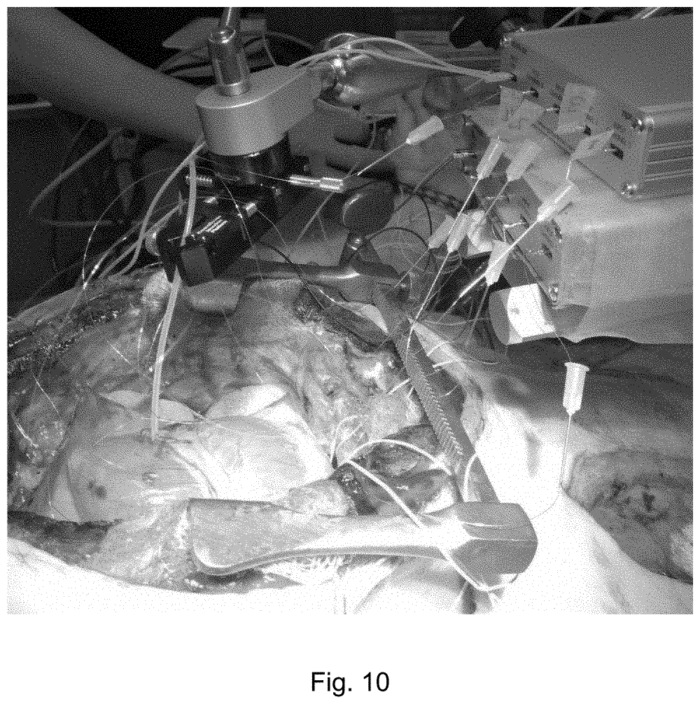

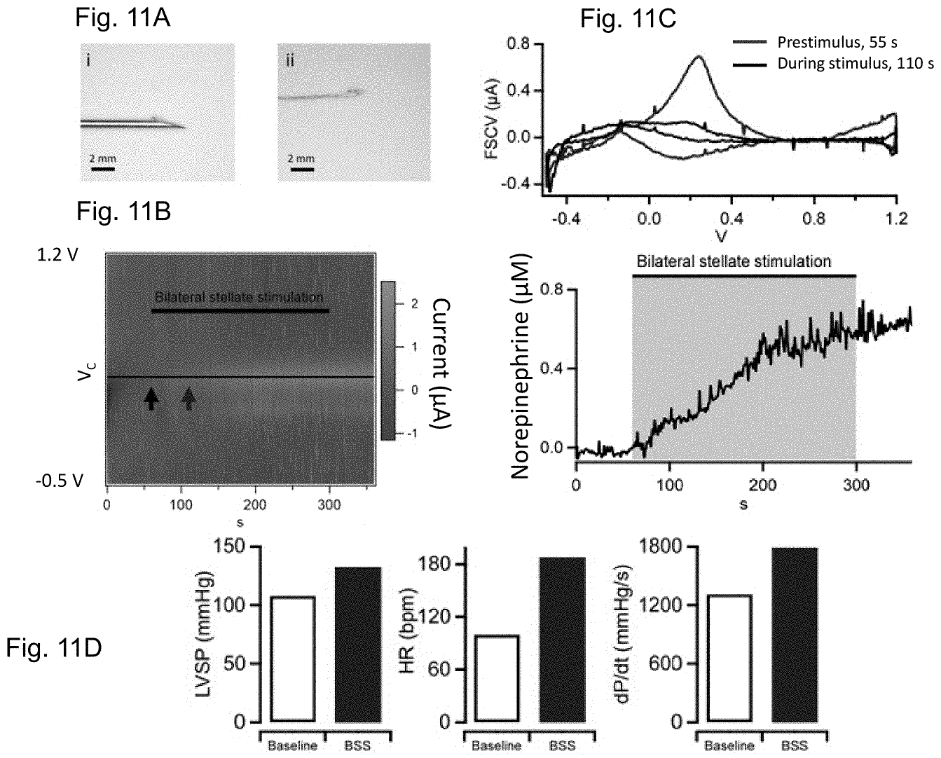

In certain embodiments, the distal end of the wire electrode comprises one or more barbs, hooks, loops, or other anchoring structures to allow for anchoring of the distal tip of the wire electrode in tissue, such as the myocardium or vessel wall. For example, in one embodiment, the distal tip of the electrode is bent backwards to produce a harpoon-like structure at the electrode tip. In certain embodiments, the wire electrode is threaded through a carrier such as needle and the wire bent backwards (FIG. 11A). The needle-wire assembly can be inserted into the tissue and the carrier withdrawn, leaving the wire electrode and its sensing element embedded within the tissue (FIG. 10). In certain embodiments, the tip of the wire electrode threaded through the carrier may have other specialized structures such as barbs on the tip to allow for anchoring of the sensor within the tissue wall when the carrier is withdrawn (FIG. 11A, ii).

In certain embodiments, the electrode is functionalized with a receptor molecule that specifically binds to a biochemical compound of interest. The receptor molecule can be any suitable molecule, small molecule, nucleic acid, amino acid, peptide, polypeptide, antibody, antibody fragment, or the like which may recognize or selectively bind the biochemical compound or compounds of interest. The receptor molecule is covalently linked to the electrode using any suitable means known in the art. In some embodiments, the receptor molecule is linked to the electrode using a linker molecule. In some embodiments, the linker molecule is any suitable linker molecule known in the art. In some embodiments, the linker molecule is a rigid linker. In some embodiments, the linker molecule is a flexible linker. In some embodiments, the linker is a cleavable linker. In some embodiments, the linker molecule is a polar molecule.

In certain embodiments, the device comprises one or more stimulatory electrodes to apply an electrical signal to the autonomic nervous system, sympathetic nervous system, parasympathetic nervous system, or cardiac nervous system. Exemplary electrodes include cuff electrodes, needle electrodes, and the like. In one embodiment, the system comprises one or more pacing electrodes suitable for application of cardiac electrical stimulation at one or more epicardial, endocardial or intramyocardial sites. In certain embodiments, one or more stimulating electrodes are used to induce release of a biochemical compound of interest (e.g., catecholamines, peptides, proteins or biomarkers) to be detected by one or more of the electrodes described herein.

In certain embodiments, the device comprises a micro-electrode array comprising a single site or a plurality of electrode sensor zones or tips suitable for placement with the tissue or vascular space either directly or directed to a site of interest by remote access.

In some embodiments, one or more of the electrodes or arrays is contained within a catheter. The catheter may be any suitable catheter as known in the art. In some embodiments, two catheters are deployed in a trans-organ arrangement (e.g., superior vena cava and aorta of the heart; coronary sinus and aorta, etc.) to measure peptide or neurotransmitter gradients across perfusion of the organ or within the organ (e.g., neuropeptide Y release in the heart).

In some embodiments, one or more electrodes comprise a semipermeable membrane encasing at least a portion of the electrode. In some embodiments, the semipermeable membrane creates a barrier between the electrode and the surrounding environment. In some embodiments, the semipermeable membrane comprises a porosity sufficiently large to allow biochemical compounds of interest to freely diffuse across the membrane. In some embodiments, the semipermeable membrane comprises a selectively semipermeable membrane. In some embodiments, the selectively semipermeable membrane selects for biochemical compounds of interest based on size, charge, polarity, composition, and the like. The semi-permeable membrane may be constructed from any suitable material known in the art.

In all embodiments, specificity of detected peptide, protein or biomarker capacitive immunoprobe signal is provided by parallel placement of a second reference electrode or sensing surface coated with a trap molecule (e.g., IgG antibody) not expected to be released or present in interstitial space, circulation or fluid compartments (e.g., actin, β-tubulin). Thus, this parallel reference signal provides a baseline for non-specific capacitance in the same space, simultaneous time and biological context of the specific trap molecule.

In some embodiments, the device of the present invention further comprises one or more controllers, connected to supply power and signals to, and to measure signals received from, electrodes of the present invention. In one embodiment, a controller is connected to a wired communication port of an electrode, but in another embodiment the connection may be implemented via a wireless link. Power may be supplied to the controller via wires or wirelessly. In certain embodiments, the device comprises an implantable controller configured to deliver and collect signals from the one or more electrodes. The implantable controller may be in wired or wireless communication with one or more external system components. For example, in certain embodiments, the implantable controller delivers and receives information from an external computing device.

In certain embodiments, the device comprises a voltage clamp circuit operably connected to the one or more electrodes. The voltage clamp circuit may be housed in one or more controllers of the device. The voltage clamp circuit may be any voltage clamp configuration, and may be positive or negative, biased or unbiased as required by the application. As understood by one skilled in the art, a voltage clamp circuit is used to fix one or more electrode potentials within pre-set limits (termed a “command potential”). In one embodiment, a system of the present invention may comprise three electrodes, including a reference electrode, a ground electrode, and a sampling or measurement electrode. In some embodiments, the reference electrode and the ground electrode may be shunted together, yielding what is effectively a two-electrode configuration. In a three electrode configuration, the potential of the reference electrode relative to ground is measured and provides the reference input for the voltage clamp of the sensor electrode. Separate ground and reference electrodes may be used in some embodiments to determine reference voltage in tissue. Such an electrode scheme may be used for example in conditions of low conductance between the sample electrode and the ground electrode—which may lead to errors in the voltage clamp and a phase offset of the obtained signals with respect to the commanded potential. Using three electrodes in such a scenario provides a more accurate voltage clamp and minimizes command potential error. This in turn leads to improved correlation between the oxidation current and the commanded potential, which provides a significantly more accurate identification of the oxidized species.

The voltage clamp circuit incorporates a feedback resistor, and the feedback resistor may have a low resistance so as to supply adequate current to the electrodes for clamp at the desired command potential. In one embodiment, the feedback resistor is a 1MΩ resistor for electrode configurations with high surface capacitance. In other embodiments, the feedback resistor is a 10MΩ resistor for higher gain and greater signal to noise measurements. In some embodiments, the device is configured to have a switchable feedback resistance, where a 1MΩ or 10MΩ feedback resistor may be selected by the operator prior to scanning. In other embodiments, the feedback resistor is a potentiometer, and the feedback resistance may be selected from a continuous range of resistances. In some embodiments, the range is from 1MΩ to 10MΩ. Such low resistances may be advantageous, for example in applications where one or more electrodes are made of platinum. In such cases, the capacitance of the electrodes will be higher, and so more current will be required to charge them.

In some embodiments, a device of the present invention comprises multiple sampling or measurement “channels” from which data is gathered simultaneously or in alternating sequence. The multiple channels may share a single reference electrode and ground electrode, or may alternatively be split among multiple reference and/or ground electrodes. Each channel has at least one distinct measurement electrode, and the various measurement electrodes may be positioned in different areas of the tissue/vasculature being measured in order to simultaneously monitor relevant concentrations across a larger area. Measurement electrodes may be substantially similar to the reference and ground electrodes, or may alternatively have a different size, shape, cross-sectional area, or material than the reference and ground electrodes. In some embodiments, the ground, reference, and measurement electrodes are all made from different materials or in different shapes. In some embodiments, the reference and ground electrodes are made from steel. In some embodiments, the reference electrodes are made from silver or silver chloride. In some embodiments, one or more of the electrodes are made from platinum.

In certain embodiments, the device comprises one or more voltage clamp amplifiers operably connected to the one or more electrodes. In certain embodiments, the one or more amplifiers are housed in one or more controllers of the device. As described herein, a voltage clamp amplifier is a circuit configured to impose a voltage across two or more electrodes while measuring the current passing through a lead connected to one or more of the electrodes. A command potential (scanning voltage waveform) is used to control the voltage on the measurement electrode with respect to the tissue voltage measured from the ground and/or reference electrodes. The command potential may be asserted by any method known in the art, including but not limited to a function generator, timing circuit, or via a digital-to-analog converter (DAC). In one embodiment, a USB controlled multi-channel DAC is used. DACs provide fast switching and voltage control, but may suffer in some cases from digital aliasing errors. That is, analog curved waveforms, for example sine waves, will look imperfect when examined at high magnification because DACs are capable only of generating a finite set of voltage values. This is particularly true if a low-resolution DAC, for example an 8-bit DAC, is used, but the effect is still present in other DACs appropriate for use in the present invention, including but not limited to a 10-bit DAC, a 12-bit DAC, a 16-bit DAC, or a 24- or 32-bit DAC. In some embodiments, the effect of the aliasing error may be mitigated by inducing a higher peak-to-peak voltage from the DAC than is required, then scaling the higher voltage down using, for example, a voltage divider and follower as known in the art. Suitable scaling factors will vary based on the capabilities of the DAC used and the voltage range required by the application, but exemplary scaling factors may be 2×, 5×, 10×, 20×, or 50×. The scaling factor in any particular device of the present invention may be fixed, or may alternatively be switchable among multiple values to allow for greater fidelity and dynamic range in command potential. In some embodiments, the voltage clamping function described above is performed by the one or more voltage clamp amplifiers. Alternatively, a single circuit or set of integrated circuits and passive components may perform both the functions of the signal acquisition and amplification and the functions of the voltage clamp as described herein.

Embodiments of the invention using DACs are advantageous because they may be easily synchronized with a corresponding analog-to-digital converter (ADC) used for data acquisition. In some embodiments, a single computer-controlled data acquisition device may be used, including one or more DACs to generate the command potential and one or more ADCs for reading data back from the device. In one embodiment, the ADCs are connected across a sensing resistor having a precise, known resistance, and record the current resulting from the oxidation or reduction of the various compounds as a voltage level across the sensing resistor.

In one embodiment, the present invention provides a biochemical compound detection device, comprising a controller and a voltage clamp amplifier. The voltage clamp circuit utilizes a three-probe strategy. The voltage command to the sensing electrode/site is set through the determination of potential drop between a voltage reference electrode and a ground electrode. The third sensing electrode is voltage clamped to a template of positive and negative voltage steps and serves as the sensor electrode whose capacitance is altered by biomolecule binding to the trap antibody/antibody fragment. This third clamped measurement circuit exists in multiples that all utilize the same reference/ground. The signal is extracted from the capacitive current supplied to clamp the electrode to a step or sinusoidal command voltage. In some embodiments, intermittent negative potential pulse is applied to the probe surface to expel the target molecule from its capture agent, providing a time-resolved signal and resetting the system/probe for further detection. This method relies on the covalent bond between antibody/antibody fragment and electrode versus the weaker non-covalent bond between antibody/antibody fragment and peptide or protein. Thus, negative potentials evoke a negative electric field at the electrode interface to electrostatically expel the peptide or protein bound to the receptor antibody/antibody fragment. In the rare case of a positively charged biomolecule, a positive potential step will serve the purpose of expulsion from the receptor molecule and the measurement step will be negative in sign. This process resets the electrode to a non-saturated state and allows for time-resolved long-term recording of the biomolecule of interest.

Exemplary command potentials for use with the present invention include but are not limited to sine waves, sawtooth waves, and square waves. The frequency of the command potential may in some embodiments be between 1 Hz and 50 Hz, or between 2 Hz and 25 Hz, or between 5 Hz and 20 Hz. Suitable amplitudes include 1.7 volts peak to peak (Vpp), 1 Vpp, 0.5Vpp, 2Vpp, or any other voltage adequate to capture concentration-dependent currents at characteristic oxidation potentials.

One exemplary embodiment of the invention is directed to the measurement of the concentration of norepinephrine, which has an oxidation voltage of approximately 400 mV, releasing two electrons per molecule when it oxidizes (FIG. 4). In this embodiment, the command potential has a Vpp of 1.7V, and a positive bias of 350 mV, resulting in a maximum voltage of +1.2V and a minimum voltage of −500 mV.

Systems of the present invention may further comprise one or more signal processing modules including but not limited to filtering, amplification, storage, and analysis modules, connected via wires or wirelessly to one or more electrodes. In some embodiments, the various signal processing modules are implemented as dedicated hardware circuitry, but the signal processing functions may also be implemented as software on a computing device. The purpose of the signal processing modules is to generate data and draw inferences from the measurements gathered from the various probes of the present invention. Filtering modules may include, but are not limited to high-pass, low-pass, or band-pass filters, Kalman filters, or any other filtering module used in the art. Amplification modules of the present invention may comprise one or more operational amplifiers or transistors, or may alternatively accomplish amplification through software means such as multiplication of analog values to add gain to some or all of the signals received. Storage modules may include any suitable means of data storage, including but not limited to hard disk drives, solid state storage, or flash memory modules.

The various sensors described herein may return measurements to a collection device as analog voltage levels, digital signals, or both. As described herein, “collection device” refers to any device capable of receiving analog or digital signals and performing at least one of: storing the data on a non-transitory computer-readable medium or, transmitting the data via a wired or wireless communication link to a remote computing device. In some embodiments, the collection device may further comprise a processor and stored instructions for performing analysis or display of the data collected. In some embodiments, the system further comprises a graphical user interface (GUI) and a display capable of presenting some or all of the data, or calculated derivatives thereof, in human readable form. The data collected may be presented as a time series kymograph, real-time display of current values, minimum or maximum values, or any other display format known in the art.

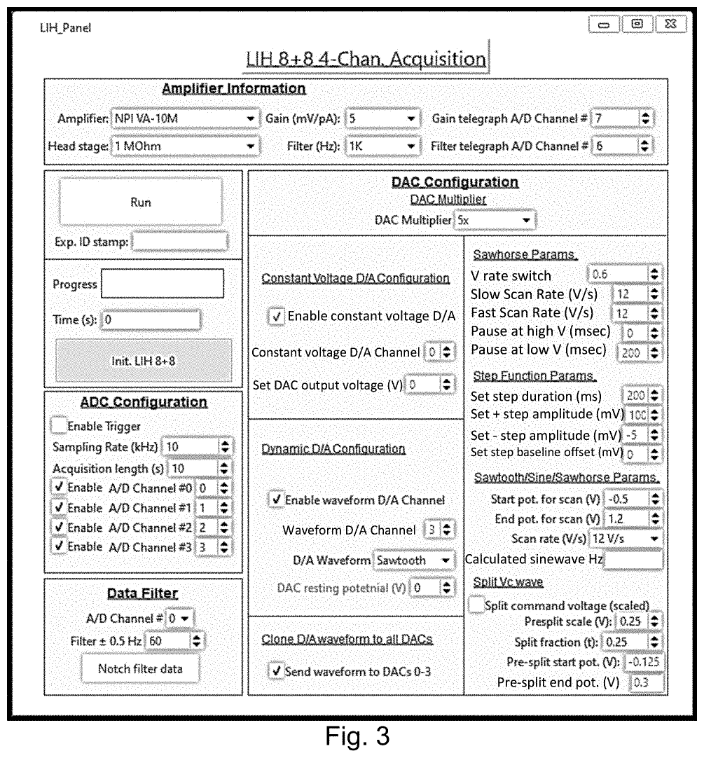

Exemplary GUIs of the present invention may include one or more controls, including Boolean, numerical, sliding, or rotary controls, for manipulation of various parameters related to systems and methods of the present invention. Examples of parameters that may be controlled by computer-implemented GUIs of the present invention include dynamic command potential and signal acquisition parameters, parameters of the command potential (including but not limited to the start potential, end potential, frequency, rate of scan, amplitude, and step size), and data measurement or acquisition parameters including but not limited to sampling granularity, sampling frequency, significant digits, and recording mode (FIG. 3). In some embodiments, a GUI of the present invention may present a set of measurements as a time-series kymograph. In other embodiments, data may be presented as a list of numerical values, or a frequency-domain graph.

Software applications of the present invention may also include one or more analysis modules, configured to perform signal or data processing steps on the raw data collected by the measurement or acquisition modules of the present invention. In one example, an analysis module may isolate oxidation- or reduction-specific signals from the capacitive currents inherent in the electrode. In another embodiment, an analysis module may perform noise detection and correction steps to remove unwanted noise from the recorded signal. In another embodiment, an analysis module may perform a frequency domain analysis of a collected time series signal, or may detect the relative position of peaks in a set of measured time-domain voltage or current values, using the position and magnitude of the located peaks to automatically determine the concentration of one or more compounds near the measurement electrode over time.

### Methods

The present invention as described herein provides methods for detecting, measuring, or monitoring the presence and abundance of one or more biochemical compounds. For example, as described herein, the present invention enables detection of one or more compounds of interest with high spatial and temporal resolution.

The method comprises the detection of any suitable biochemical compounds of interest, including, but not limited to neurotransmitters, proteins, peptides, nucleic acid molecules, hormones, and the like.

In some embodiments, the method is used for the detection of specific peptides in the heart, including but not limited to Enkephalins, Neuropeptide Y, substance P, calcitonin gene-related peptide (CGRP), and brain natriuretic peptide (BNP). In certain embodiments, the method is used for the detection of neurotransmitters, including, but not limited to catecholamines, such as norepinephrine, epinephrine, and acetylcholine.

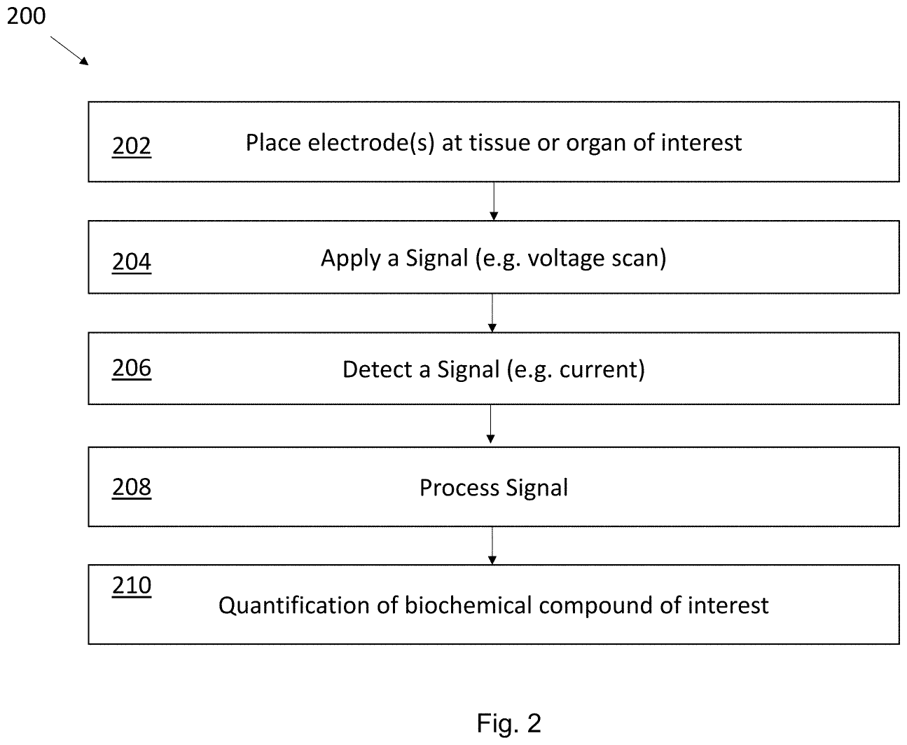

Referring now to FIG. 2, an example process 200 for detecting the presence and abundance of a biochemical compound of interest is shown. One or more steps of process 200 may be implemented, in some embodiments, by one or more components of the system and device, as described herein. In some embodiments, as depicted in block 202, the method comprises placing one or more electrodes, as described herein, within a region of interest. The one or more electrodes may be placed in any suitable location to detect the biochemical compounds of interest.

In some embodiments, the region of interest is one or more locations within the myocardium. In some embodiments, the region of interest is adjacent to an organ or tissue of interest. In some embodiments, the region of interest is adjacent to one or more nerves, nerve divisions, ganglia or regions of a nerve of interest. In some embodiments, the region of interest is within one or more nerves, ganglia, nerve divisions and the like. In some embodiments, the one or more electrodes are placed into vascular space in proximity to the organ or tissue of interest. In some embodiments, the one or more electrodes is placed into interstitial space in proximity to an organ or tissue of interest. In some embodiments, the one or more electrodes are placed into a chamber of the heart, for instance the right atrium, the right ventricle, the left atrium, and/or the left ventricle. In some embodiments, the one or more electrodes are placed into a blood vessel, for example, inferior vena cava, superior vena cava, coronary sinus, coronary artery, coronary vein, ascending aorta, aorta, pulmonary artery, pulmonary vein, great veins of the heart, a peripheral vein, a peripheral artery and the like. In some embodiments, the one or more electrodes are placed into the pericardial space.

For example, in certain embodiments, one or more electrodes are placed in the atrial myocardium, ventricular myocardium, vascular space of the heart, coronary sinus of the heart, left ventricle, right ventricle, left atrium, right atrium, epicardial fat pad, pericardial fat pad, aorta, pulmonary vein, pulmonary artery, vena cava, or the like. In certain embodiments, one or more electrodes can be placed within a neural structure, including at a neural structure of the autonomic nervous system, such as at one or more of a peripheral nerve, the intrathoracic ganglia, stellate ganglia, autonomic ganglia, nodose ganglia, dorsal root ganglia, petrosal ganglia, or sensory ganglia. In various embodiments, the method comprises placement of one or more electrodes at different locations within the autonomic nervous system and/or heart to detect regional differences in the abundance of one or more biochemical compounds of interest. In some embodiments, the electrodes are placed in the airways/alveoli of the lung.

In one embodiment, the method comprises inserting one or more wire electrodes into a region of interest. For example, in one embodiment, the method comprises inserting a wire electrode through the distal tip of a needle (FIG. 11A), inserting the needle through cardiac tissue, and withdrawing the needle, thereby leaving the electrode within the tissue (FIG. 10). In some embodiments, prior to insertion of the needle, the wire is advanced past the needle tip, and the wire is bent backwards along the shaft of the needle forming a harpoon-like shape, enabling the electrode to remain in the tissue while the needle is withdrawn. In some embodiments, the distal tip of the electrode comprises one or more anchoring structures, as described elsewhere herein, thereby allowing the electrode to remain in the tissue while the needle is withdrawn.

In some embodiments, as depicted in block 204, the method of the invention further comprises applying a signal to one or more electrodes. In certain embodiments, the method comprises the use of voltammetry, including, but not limited to fast scanning cyclic voltammetry (FSCV), potential step voltammetry, linear sweep voltammetry, cyclic voltammetry, square wave voltammetry, staircase voltammetry, anodic or cathodic stripping voltammetry, adsorptive stripping voltammetry, alternating current voltammetry, rotated electrode voltammetry, normal or differential pulse voltammetry, chronoamperometry, and chronocoulometry. In some embodiments, an FSCV signal is applied to one or more electrodes.

In certain embodiments (FIG. 2, step 204), a control unit or controller is configured to deliver a signal to one or more electrodes. The signal may comprise a constant voltage or a specific pattern of variable voltage. For example, in certain embodiments, the method comprises delivering a pattern of increasing and decreasing voltages (i.e., voltage scanning) in a step, triangular, sinusoidal, saw tooth, or any other suitable pattern. In FSCV applications, the method comprises rapidly increasing and decreasing the voltage at the electrode tip. In certain embodiments, the method comprises administering a cyclic voltage signal, where the applied pattern of voltage is repeated for a defined duration or number of periods. In some embodiments, the signal is applied at a frequency of less than 1 Hz, 1 Hz to 50 Hz, or greater than 50 Hz. In one embodiment, the signal is applied at a frequency in the range of about 1 Hz to 50 Hz.

In certain embodiments, the delivered voltage scans between a minimum voltage of about −5V to −200 mV and a maximum voltage of about 200 mV to 5V. In one embodiment, the delivered voltage scans between about −500 mV to about 1.2V. In one embodiment, the voltage scans can be delivered at rate of about 1-50 V/s. In one embodiment, the voltage scans can be delivered at rate of about 5-20 V/s.

In some embodiments, as depicted in block 206, the method comprises detecting a signal from one or more electrodes. For example, in certain embodiments, the method comprises detecting a current in response to the delivered voltage signal. In certain embodiments, the method comprises measuring a current using the same electrode that was used to deliver the voltage. In certain embodiments, the method comprises detection of current indicative of the oxidation and/or reduction of the biochemical compound of interest. As described elsewhere herein, the delivered voltage scan results in the oxidation and reduction of biochemical compounds in the vicinity of the electrode sensor zone which produces a current overlaid on the background current detected by the electrode.

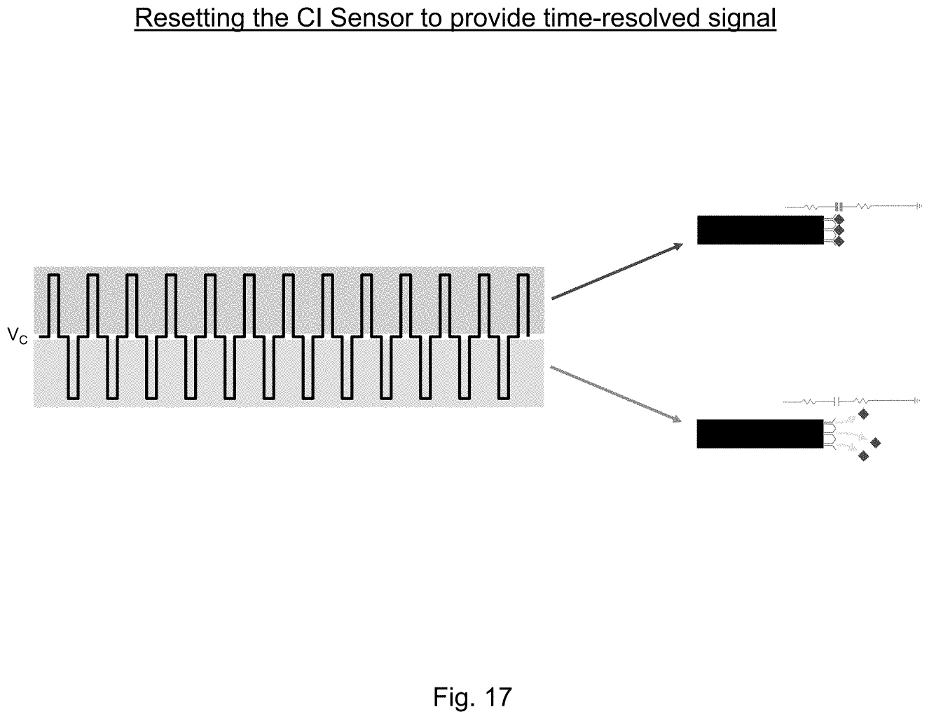

In certain embodiments, where the electrode is functionalized with a receptor molecule, the presence of a biochemical compound of interest that specifically binds to the receptor molecule is observed by detecting a change in the capacitance of the electrode. For example, in certain aspects, binding of the compound of interest to the receptor molecule increases or decreases the native capacitance of the electrode. The change in capacitance can be measured in any suitable manner. For example, in certain embodiments, the capacitance of the electrode can be measured by delivering voltage steps to the electrode and measuring the time constant and charge amplitude of the electrode, thereby enabling the calculation of the capacitance, a parameter that changes upon detection and binding of the molecule of interest to the capture agent (FIG. 14B). In one embodiment, the capacitance of the electrode can be measured by measuring a current or a change in a current. In other embodiments, capacitance of single equivalent circuits are measured in a frequency-domain analysis allowing for spectral un-mixing of multiple signals on a single electrode, each specific for a single molecule of interest. In conventional capacitive immunosensing and immune-based techniques (i.e. ELISA), the signal saturates as the antibody or capture agent binds its target molecule (protein) making time-resolved measures of dynamic levels of the protein or hormone impossible. In an embodiment of the present invention, the probe is continually reset during the recording to avoid saturation and to allow dynamic, time-resolved measures of the target molecule (FIG. 17). This is accomplished through an intermittent negative potential pulse to expel the target molecule from its capture agent, providing a time-resolved signal and resetting the system/probe for further detection (FIG. 17). Resetting allows for continuous or sequential biomolecule recording over time frames from seconds up to a day or longer.

Multiple biomolecules can be achieved from the same, immediately adjacent or remote sites. In one such iteration, such an embodiment would be designed by attaching more than one receptor molecule (e.g., antibody) to the sensor zone of the electrode, thus allowing for the measure of multiple molecules of interest simultaneously, with each signal respectively separated in a frequency-domain analysis. In another interaction, such an embodiment would be designed by attaching specific trap molecules to different electrode sites along a single shaft linear micro-array electrode or to closely adjacent shafts of a 2D microarray or 3D microarray.

In some embodiments, as depicted in block 208, the method comprises processing one or more signals detected from the one or more electrodes. In certain embodiments, a control unit or controller may process the signal so that the detected signal is recorded or displayed as a voltage, current, capacitance, or any other relevant parameter.

In certain embodiments, as depicted in block 210, the method comprises processing the signal to produce a voltammogram of detected current as a function of voltage. In one embodiment, a voltammogram is produced by subtracting baseline current from the detected current, in response to an applied voltage scan, thereby producing the oxidation current induced by the biochemical compound of interest. In certain embodiments, one or more characteristics of the voltammogram are used to identify the compound. For example, as shown in FIG. 6A and FIG. 6B, the oxidation of norepinephrine produces a single peak, while the oxidation of epinephrine produces two peaks. Therefore, in certain embodiments, the method comprises comparing the voltammogram with a standard or reference voltammogram to identify the one or more detected compounds.

In certain embodiments, the method comprises quantifying the amount of the biochemical compound of interest. For example, in certain embodiments, the method comprises identifying the peak current, where the amplitude of the peak current can be used to calculate the concentration of the compound of interest. For example, in certain embodiments, a standard curve or calibration curve is used to calculate the concentration of the compound of interest. The standard curve or calibration curve can be based upon the peak amplitudes detected in the in vitro or ex vivo detection of known concentrations of the compound of interest. Use of a standard curve to calculate the concentration of detected norepinephrine and epinephrine is shown in FIG. 7.

In some embodiments, the method comprises recording and storing the detected signal. In certain embodiments, the method comprises recording and storing the detected signal and the applied signal (e.g., voltage scan).

In some embodiments, the detected signal may be processed in order to determine trends in the detected signal. For example, the detected signal may be processed as voltage with respect to time, as voltage with respect to current, as current with respect to time, and the like, as known in the art. In some embodiments, calibration curves may be computed from the detected signal. For example, the signal (i.e. current, voltage, capacitance, etc.) that is detected when the sensor is placed in proximity to known concentrations of a biological compound of interest may be used in order to calibrate the detected signal to one or more known concentrations. In some embodiments, the computed calibration curves may be used in order to quantify the concentration of an unknown amount of a biological compound of interest. In some embodiments, the controller automatically generates calibration curves that may be used to compute concentrations of unknown amounts of biological compounds. In some embodiments, the calibrated concentration of a detected biological compound may be displayed on the user interface of the controller. In some embodiments, the sensor may be calibrated in order to determine whether a biological compound is detected or not. In some embodiments, the detected signal and/or processed signal may be stored by the controller. In some embodiments, the detected signal and/or processed signal may be transferred by means known in the art to an external device.

In certain embodiments, the present invention provides a method of detecting or monitoring the level of a biochemical compound of interest, such as a neurotransmitter or protein or peptide of interest, in response to one or more cardiac stressors or other stimulation. In one embodiment, the one or more cardiac stressors comprises transient reductions or increases in cardiac preload (venous return). In one embodiment, the one or more cardiac stressors comprise a transient increase or decrease in cardiac afterload (arterial blood pressure). In one embodiment, the one or more cardiac stressors comprise increases or decreases in sympathetic efferent inputs to the heart. For example, in certain embodiments, a change in sympathetic efferent inputs to the heart is achieved by stimulation or local block of intrathoracic sympathetic projections to the heart. In certain embodiments, a change in sympathetic efferent inputs to the heart is achieved by stimulation or block of the dorsal aspect of the spinal cord. In one embodiment, the one or more cardiac stressors comprise increases or decreases in parasympathetic efferent inputs to the heart. In certain embodiments, a change in parasympathetic efferent inputs is achieved by stimulation or local block of parasympathetic efferent projections to the heart. In one embodiment, the one or more cardiac stressors comprises increases or decreases in autonomic control of the heart. For example, in one embodiment a change in the autonomic control of the heart is achieved by stimulation or local block of intrinsic cardiac ganglia. In one embodiment, the one or more cardiac stressors comprise increases or decreases in cardiac afferent input. For example, in one embodiment a change in the cardiac afferent input is achieved by stimulation or local block of intrathoracic sensory input to autonomic ganglia. In one embodiment, a change in afferent input is achieved by stimulation or block of nodose afferent neurons. In one embodiment, a change in afferent input is achieved by stimulation or block of dorsal root ganglia. In one embodiment, the more or more cardiac stressors comprises cardiac pacing. Such cardiac pacing may be from electrodes placed on or in the atrium, ventricles or both. In one embodiment, the pacing may be condition-test pacing where a set of conditioned pace beats is followed by one or more pace stimuli of shorter inter-pace interval. In one embodiment, the pacing may be decremental with progressive decreases in inter-pace intervals. In one embodiment, the pacing may be burst type pacing with burst frequencies between 1 to 10 Hz. In one embodiment, the pacing may be synchronized to cardiac electrical activity to deliver a single or multiple pulses at cycle lengths less than the basal heart rate cycle length; such pacing stimuli modeling premature atrial and ventricular electrical events. In one embodiment, chemicals that modulate cardiomyocyte or neural activity may be placed on the heart or injected into the vascular space. In one embodiment, changes in ventilation may be used as a transient cardiopulmonary stress. In one embodiment, changes in ventilation may include one or more of the following, changes in ventilation rate, ventilation tidal volume, outflow pressure, and inflow gas mixture.

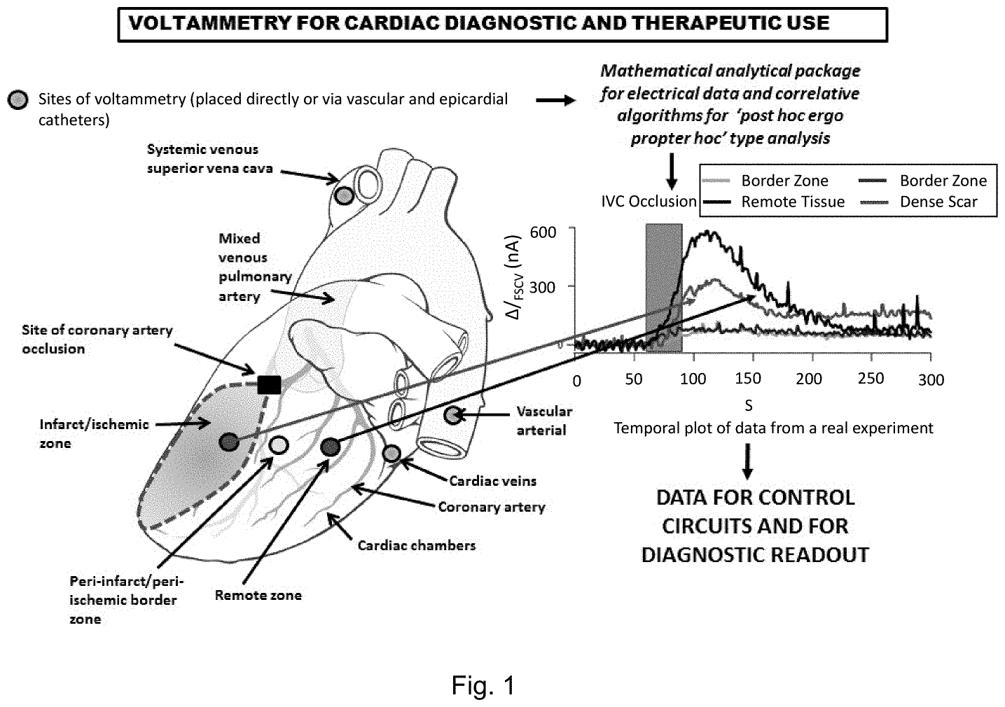

In one aspect, the invention relates to a method for monitoring cardiac or cardiopulmonary autonomic function or dysfunction, comprising inserting one or more electrodes into a myocardium and applying a voltage scan (e.g. a FSCV signal) to measure neurotransmitter (e.g., catecholamine) levels in the vicinity of the sensor zone of the electrode. In certain embodiments, the one or more electrodes are placed into the atrial myocardium or into the ventricular myocardium. The electrode or electrodes may be placed from vascular access or epicardial access. FIG. 1 illustrates an exemplary distribution of interstitial recording electrodes placed into the ventricles. However, the present invention is not limited to the particular distribution depicted in FIG. 1.

In another aspect, the invention relates to a method for monitoring cardiac or cardiopulmonary autonomic function or dysfunction, comprising inserting a catheter-based electrode into vascular space of a heart, and applying a voltage scan (e.g., a FSCV signal) to measure neurotransmitter (e.g., catecholamine) content in the vicinity of the catheter-based electrode. In certain instances the catheter-based electrode is an FSCV sensor. In one embodiment, the catheter-based electrode is placed in a coronary sinus of the heart to measure neurotransmitter levels at the immediate venous outflow from the heart. In one embodiment, the catheter-based electrode is placed in the great veins of the heart to measure neurotransmitter (e.g. catecholamine) levels at the inflow to the heart. In one embodiment, the catheter-based electrode is placed in the left ventricle of the heart or the aorta to measure neurotransmitter (e.g., catecholamine) levels before entry to the coronary vasculature of the heart. In one embodiment, the catheter-based electrode is placed in the right ventricle of the heart or a pulmonary artery to measure neurotransmitter (e.g., catecholamine) levels before entry to the pulmonary vasculature of the heart. In one embodiment, the catheter-based electrode is placed in the left atrium or pulmonary veins to measure neurotransmitter (e.g. catecholamine) levels after exit from the pulmonary circulation. In one embodiment, a plurality of catheter-based electrodes are placed in one or more of a coronary sinus, cardiac chambers, vena cava or aorta of the heart to measure trans-cardiac neurotransmitter (e.g., catecholamine) levels. In one embodiment, a plurality of catheter based electrodes are placed into one for more of the right atria, right ventricle or pulmonary artery (e.g. inflow to pulmonary circuit) and pulmonary veins or left atria (e.g. outflow from pulmonary circuit) to measure trans-pulmonary neurotransmitter (e.g. catecholamine) levels. In one embodiment, the catheter-based electrode is placed directly in blood. In one embodiment, the method comprises inserting a catheter-based electrode into vascular space and applying a voltage scan (e.g., FSCV signal) to measure neurotransmitter (e.g. catecholamine) content in the vicinity of the recording sensor in response to one or more cardiac stressors or stimulation, as described above. In one embodiment, the local, transcardiac and transpulmonary basal neurotransmitter (e.g. catecholamine) levels are assessed in the vascular compartment. In one embodiment, the local, transcardiac and transpulmonary neurotransmitter (e.g. catecholamine) levels are assessed in the vascular compartment in response to one or more cardiac stressors or stimulation, as described above.

In one embodiment, a semi-permeable membrane is placed between the catheter-based electrode and blood. For example, in certain embodiments, the catheter-based electrode comprises a semi-permeable membrane. In one embodiment, the pore size of the semi-permeable membrane is sufficient to allow passage of neurotransmitter (e.g., catecholamine) from the blood to the vicinity of the electrode.

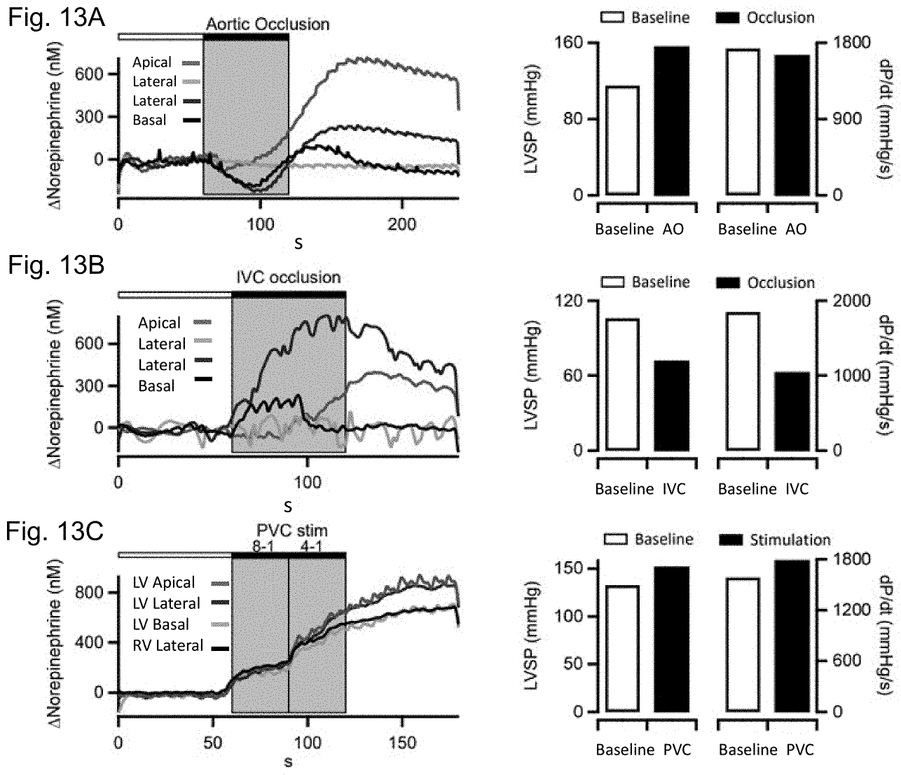

In another aspect, the present invention relates to a method of assessing regional differences in autonomic control of regional cardiac function or dysfunction. In one embodiment, the method comprises inserting multiple electrodes into a myocardium of a heart and applying a voltage scan (e.g., an FSCV signal) to measure regional levels in a local vicinity of a sensor zone of the electrode. In one embodiment, regional basal neurotransmitter (e.g., catecholamine) levels are assessed. In one embodiment, regional neurotransmitter (e.g., catecholamine) levels are assessed in response to one or more cardiac stressors or stimulation, as described above. FIG. 13B and FIG. 1 depict representative catecholamine release profiles into the ventricular interstitium in response to a decrease in preload produced by transient occlusion of the inferior vena cava.

In another aspect, the present invention provides a method for measuring neurotransmitter (e.g., catecholamine) levels in the peripheral blood, comprising inserting an electrode into a blood vessel and applying a voltage scan (e.g., a FSCV signal) to measure neurotransmitter (e.g., catecholamine) levels in the vicinity of a sensor zone of the electrode. In one embodiment, the electrode is placed into a peripheral artery. In one embodiment, the electrode is placed into a peripheral vein. In one embodiment, the electrode is a catheter-based electrode. In one embodiment, the electrode is placed from vascular access. In one embodiment, a semi-permeable membrane is placed between the catheter-based electrode and blood. For example, in certain embodiments, the catheter-based electrode comprises a semi-permeable membrane. In one embodiment, the pore size of the semi-permeable membrane is sufficient to allow passage of neurotransmitter (e.g., catecholamine) from the blood to the vicinity of the electrode.

In one aspect, the present invention provides a method for monitoring cardiac or cardiopulmonary autonomic function or dysfunction, comprising inserting one or more functionalized electrodes (e.g., capacitive immunosensors) into a myocardium and applying a signal (e.g., voltage) to the functionalized electrode to measure the level of a protein or peptide of interest in the local vicinity of the sensor zone of the functionalized electrode. In certain embodiments, the one or more functionalized electrodes are placed into the atrial myocardium, into the ventricular myocardium or both. The functionalized electrode or electrodes may be placed from vascular access or epicardial access.

In another aspect, the invention relates to a method for monitoring cardiac or cardiopulmonary autonomic function or dysfunction, comprising inserting a catheter-based functionalized electrode into vascular space of a heart, and applying a signal (e.g., voltage) to measure the level of a protein or peptide of interest in the vicinity of the catheter-based functionalized electrode. In one embodiment, the catheter-based functionalized electrode is placed in a coronary sinus of the heart to measure the level of a protein or peptide of interest at the immediate venous outflow from the heart. In one embodiment, the catheter-based functionalized electrode is placed in the great veins of the heart to measure the level of a protein or peptide of interest at the inflow to the heart. In one embodiment, the catheter-based functionalized electrode is placed in the left ventricle of the heart or the aorta to measure the level of a protein or peptide of interest before entry to the coronary vasculature of the heart. In one embodiment, the catheter-based functionalized electrode is placed in the right ventricle of the heart or a pulmonary artery to measure the level of a protein or peptide of interest before entry to the pulmonary vasculature of the heart. In one embodiment, the catheter-based functionalized electrode is placed in the left atrium or pulmonary veins to measure the level of a protein or peptide of interest after exit from pulmonary vascular circuit. In one embodiment, a plurality of catheter-based functionalized electrodes are placed in one or more of a coronary sinus, cardiac chambers, vena cava or aorta of the heart to measure the trans-cardiac level of a protein or peptide of interest. In one embodiment, a plurality of catheter-based functionalized electrodes are placed in one of more of a great vein, right atria, right ventricle, pulmonary artery, pulmonary vein, left atria or left ventricle to measure the trans-pulmonary level of a protein for peptide of interest. In one embodiment, the catheter-based functionalized electrode is placed directly in blood. In one embodiment, the method comprises inserting a catheter-based functionalized electrode into vascular space and applying a signal (e.g., voltage) to the level of a protein or peptide of interest in the vicinity of the recording sensor in response to one or more cardiac stressors or stimulation, as described above. In one embodiment, the local, transcardiac or transpulmonary basal level of a protein or peptide of interest are assessed in the vascular compartment. In one embodiment, the local, transcardiac and/or transpulmonary levels of a protein or peptide of interest are assessed in the vascular compartment in response to one or more cardiac or pulmonary stressors or stimulation, as described above.

In one embodiment, a semi-permeable membrane is placed between the catheter-based functionalized electrode and blood. For example, in certain embodiments, the catheter-based functionalized electrode comprises a semi-permeable membrane. In one embodiment, the pore size of the semi-permeable membrane is sufficient to allow passage of a protein or peptide of interest from the blood to the vicinity of the functionalized electrode.

In one embodiment, the present invention provides a method of assessing a regional difference in autonomic control of regional cardiac function. In one embodiment, the method comprises inserting a plurality of functionalized electrodes into the myocardium, autonomic ganglia, or sensory ganglia. In one embodiment, the method comprises applying functionalized electrodes to measure the regional levels of one or more proteins or peptides of interest in the local vicinity of the sensor zone of each functionalized electrode. In one embodiment, regional cardiac interstitial basal protein or peptide transmitter levels are assessed. In one embodiment, regional cardiac interstitial protein or peptide transmitter levels are assessed in response to cardiac stressors, pulmonary stressors or stimulation as described above. In one embodiment, interstitial protein or peptide levels are assessed in one or more of intrathoracic autonomic, stellate, nodose, dorsal root, and/or petrosal ganglia at baseline and in response to cardiac stressors, pulmonary stressors or stimulation as described above.

In another aspect, the present invention provides a method for measuring the level of a protein or peptide of interest in the peripheral blood, comprising inserting one or more functionalized electrodes into a blood vessel and applying a signal (e.g., voltage) to measure the levels of one or more proteins or peptides of interest in the vicinity of the sensor zone of each functionalized electrode. In one embodiment, the electrode is placed into a peripheral artery. In one embodiment, the electrode is placed into a peripheral vein. In one embodiment, the functionalized electrode is a catheter-based functionalized electrode. In one embodiment, the functionalized electrode is placed from vascular access. In one embodiment, a semi-permeable membrane is placed between the catheter-based functionalized electrode and blood. For example, in certain embodiments, the catheter-based functionalized electrode comprises a semi-permeable membrane. In one embodiment, the pore size of the semi-permeable membrane is sufficient to allow passage of a protein or peptide of interest from the blood to the vicinity of the functionalized electrode.

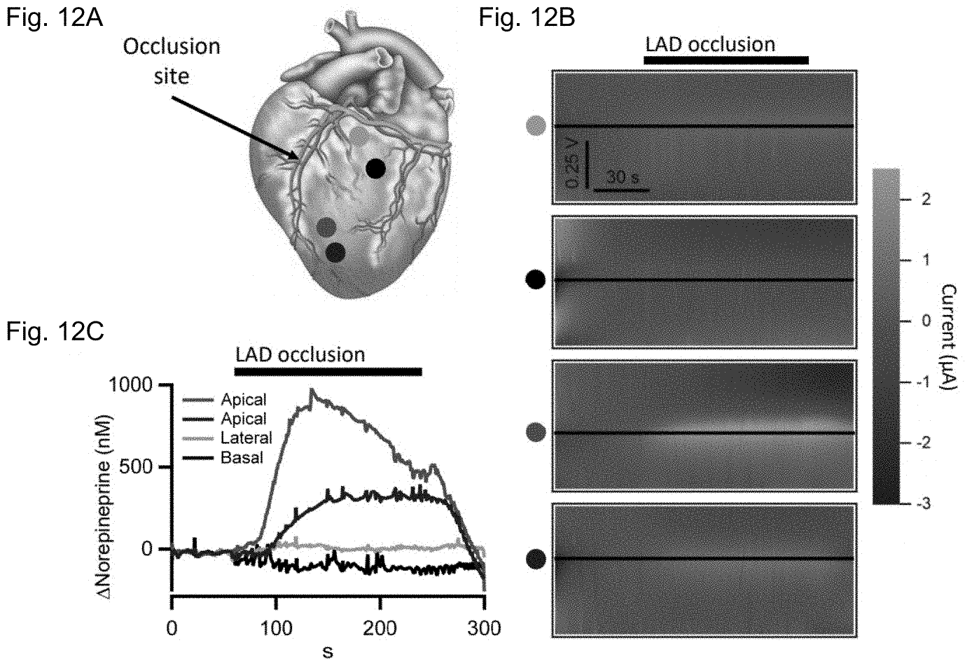

In certain embodiments, the present invention provides a method for detection of a cardiac defect or cardiac dysfunction in a subject by measuring one or more biochemical compounds. For example, in certain embodiments, the method comprises detecting a cardiac defect or cardiac dysfunction using one or more of the electrodes described herein to detect a neurotransmitter (e.g., catecholamines) or protein or peptide of interest. For example, as described herein, LAD occlusion resulted in the observation of increased concentrations of norepinephrine measured using voltammetry. Thus, the methods of the present invention can be used to detect cardiac dysfunction including, but not limited to, myocardial infarction, great vessel occlusion and modulation of autonomic inputs to the heart In certain embodiments, the ability to measure regional differences in catecholamines (FIG. 1), in addition to other neuromodulators and hormones, provides greater insights into normal and abnormal function of the neural-heart interface that can be predictive of adverse outcomes, including potential for arrhythmias and heart failure. In certain embodiments, the ability to measure regional differences in catecholamines (FIG. 12A through FIG. 13C) in addition to other neuromodulators and hormones, provides a methodology to rapidly assess efficacy to therapeutic interventions. In certain embodiments, the ability to measure regional differences in the vascular compartment for catecholamines in addition to other neuromodulators and hormones provides greater insight into relevant biomarkers indicative of susceptibility to cardiac pathology and the progression of the cardiovascular disease process.

In one embodiment, the present invention provides a method for treating or preventing a cardiac defect or dysfunction in a subject, based upon the detection of one or more biochemical compounds. In certain embodiments, the method comprises treating the subject with at least one therapeutic element upon the detection of an aberrant level or pattern of one or more biochemical compounds. In certain embodiments, the treatment may include the administration of a drug, compound or other chemical or biological material. In certain embodiments, the treatment may include administration of an electrical stimulus or other forms of energy including, but not limited to, focal temperature changes, radiofrequency, electromagnetic radiation, infrared radiation, or ultrasound, to one or more regions of the heart, including any myocardial tissues or any intrinsic neurons associated therewith. In certain embodiments, the treatment may be administered to extracardiac nexus points including, but not limited to the intrathoracic ganglia, the vagosympathetic trunk, and the spinal cord.

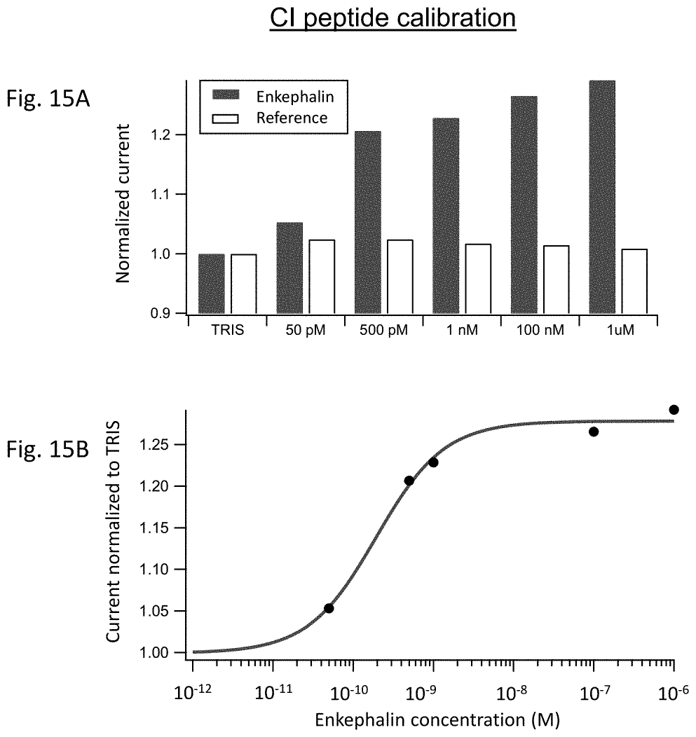

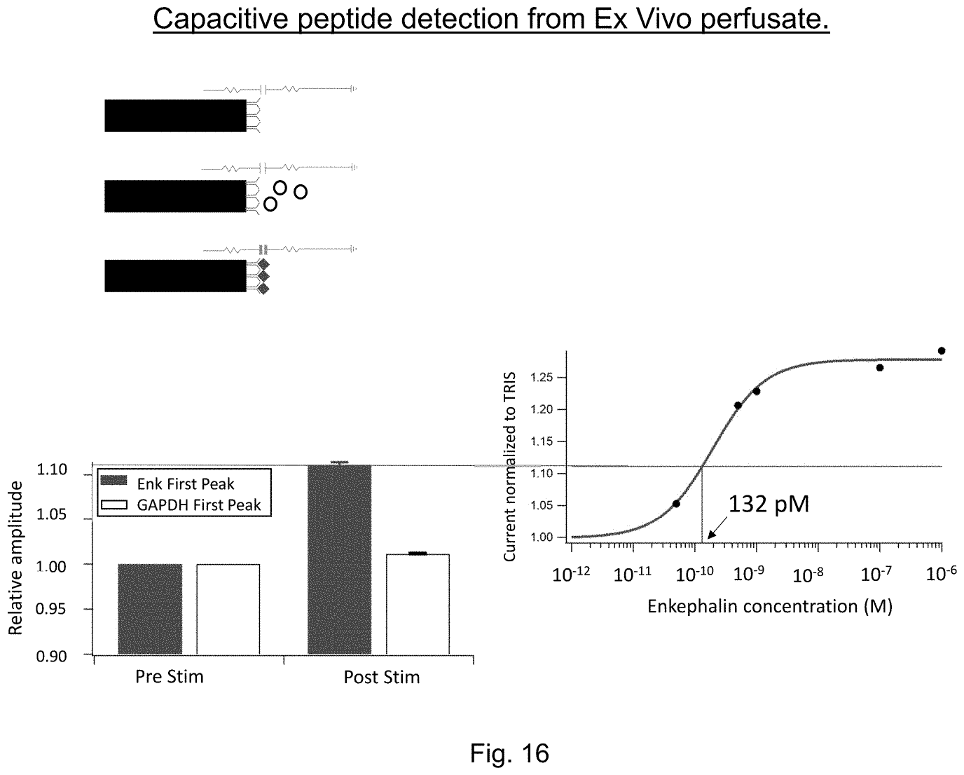

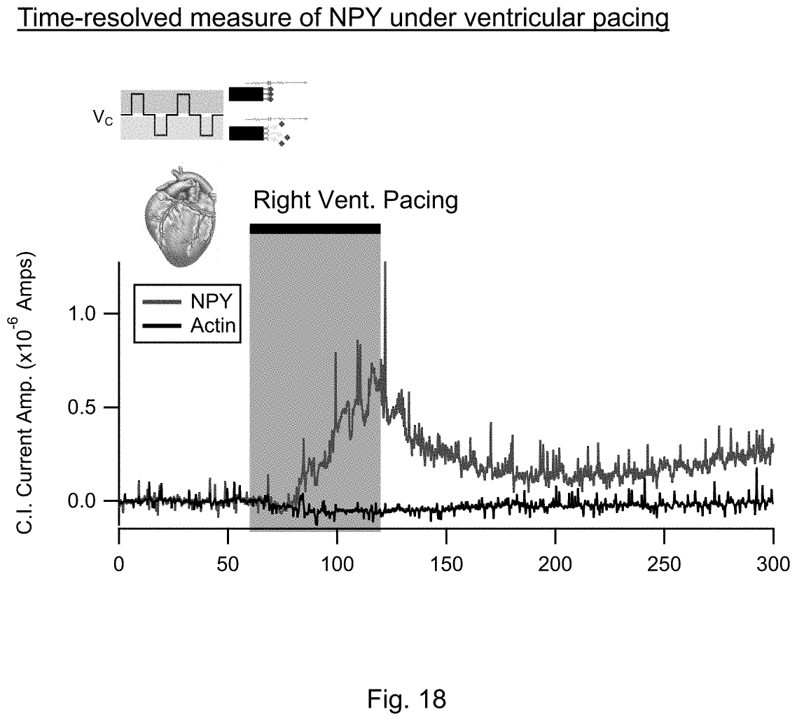

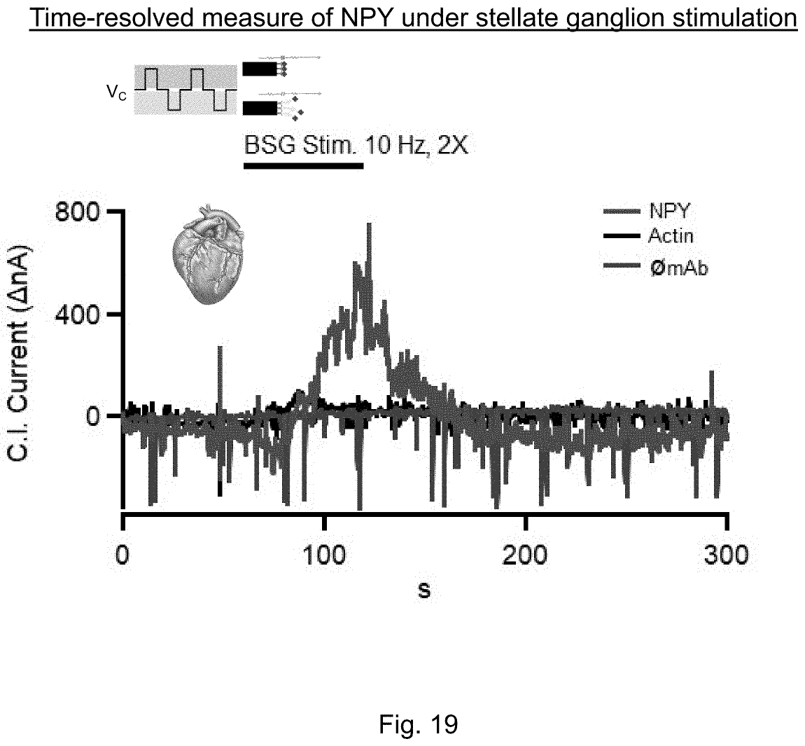

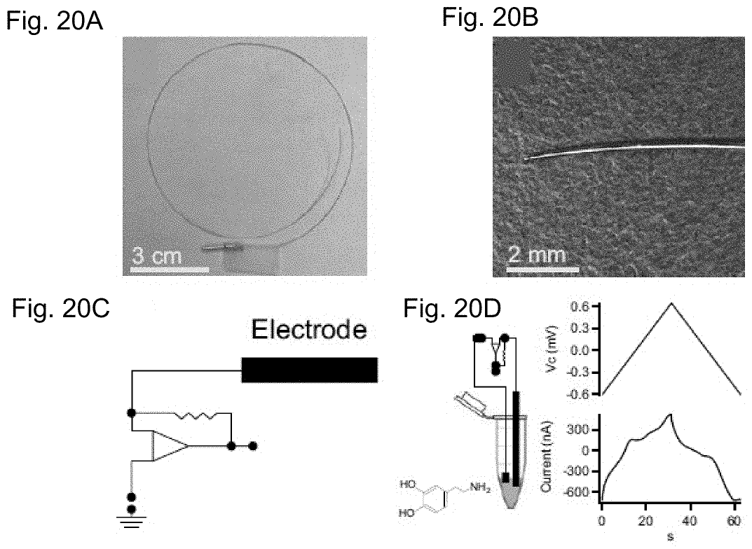

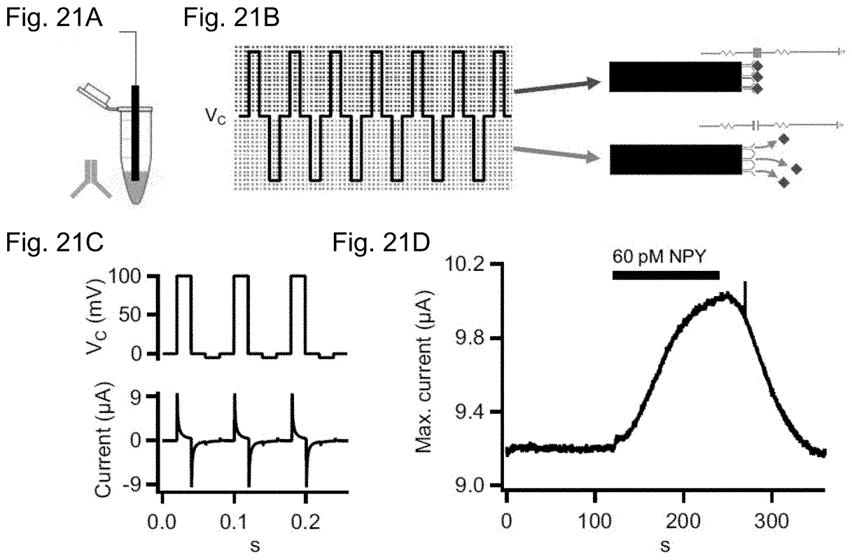

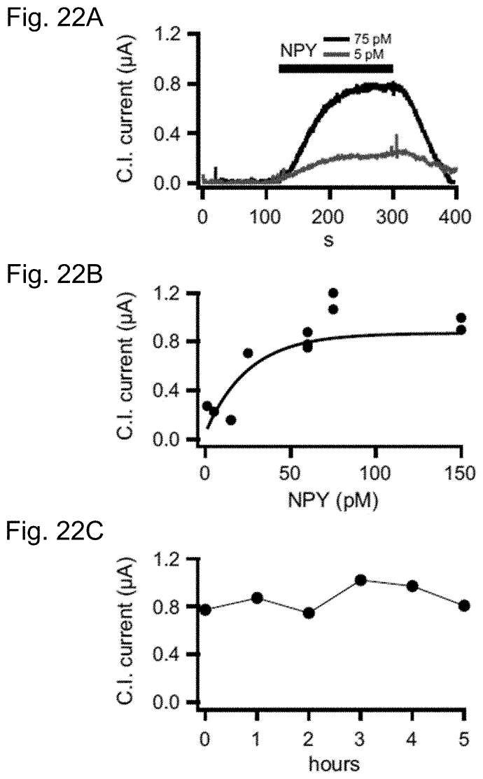

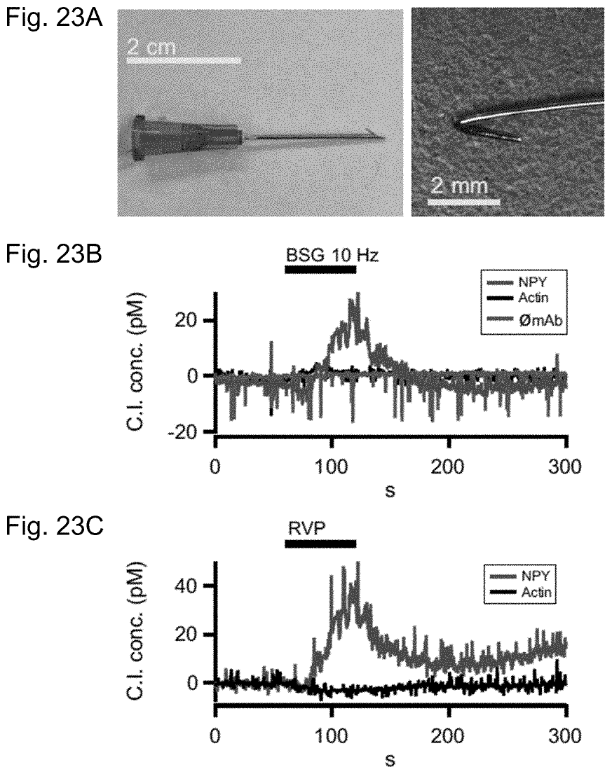

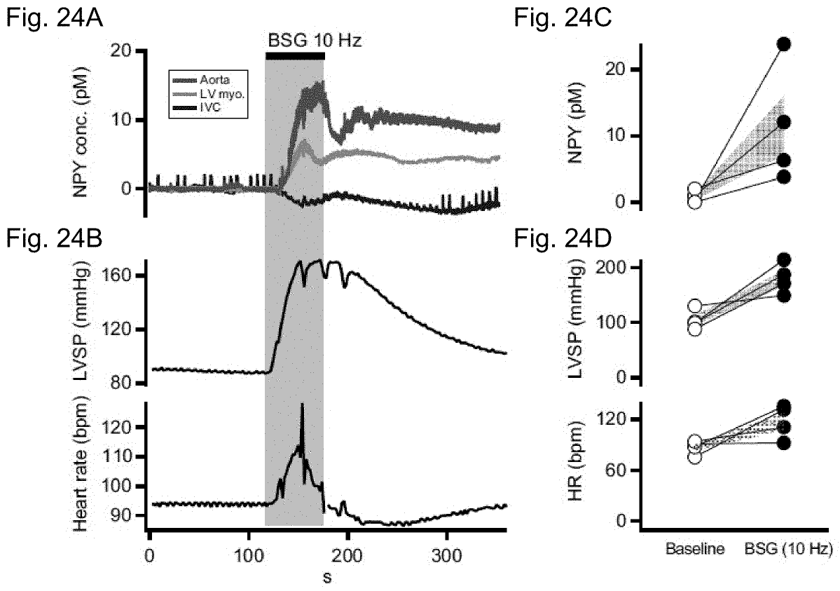

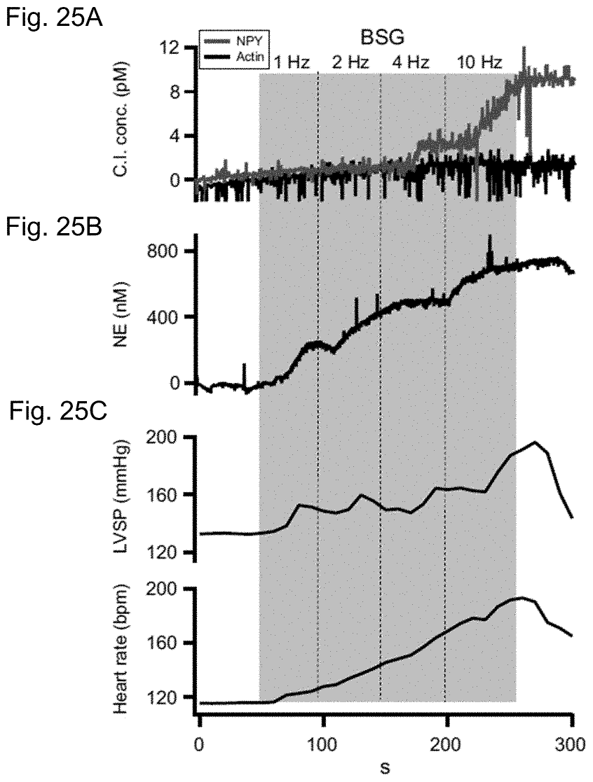

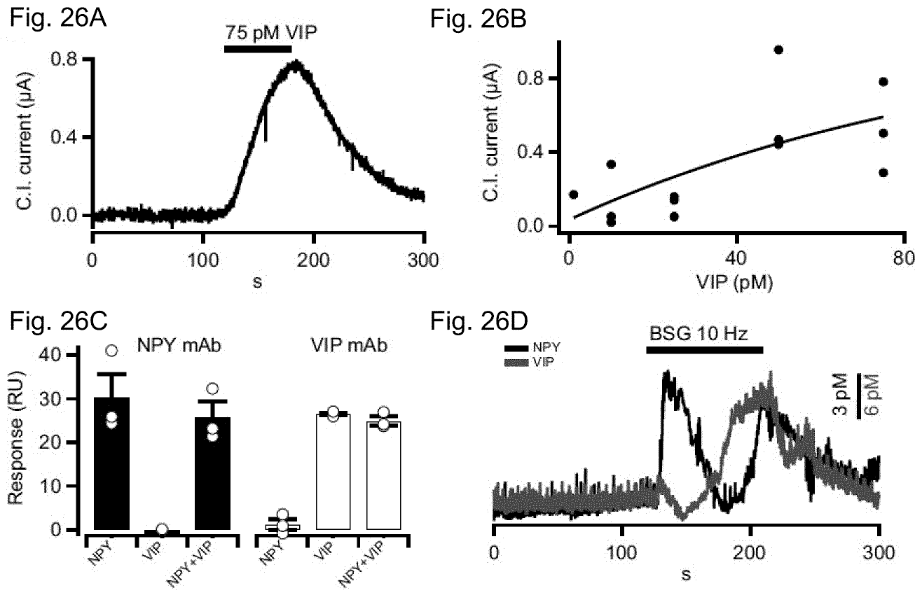

In one embodiment, the present invention provides a method for detecting a biochemical compound, comprising inserting one or more detection electrodes and complementary negative control electrodes in one or more locations selected from the group consisting of: a tissue/organ, peripheral blood vessel, lymphatic vessel/node, and extravascular fluid compartment, wherein at least one electrode comprises a receptor molecule that specifically binds the biochemical compound; and detecting a change in the capacitance of the electrode thereby indicating the presence of the biochemical compound. Capacitance is determined by measuring the current to charge the electrode to a step voltage command by the relationship of Q=C*V where Q=charge, C=capacitance, and V=voltage. Q per unit time represents current and is the measured parameter (FIG. 11B). Current amplitudes are then calibrated against a standard curve (FIG. 16) specific for the antibody or trap molecule (FIG. 15A, FIG. 15B) for quantitative analysis. Alternatively, capacitance can be determined in frequency domain through impedance analysis by use of a phase lock-in amplifier and measuring the phase offset between command voltage and measured current followed by capacitance deconvolution. Examples of this approach are provided in the context of detection of neuropeptide Y (NPY) in an open chest pig model (FIG. 18 and FIG. 19). Electrodes functionalized with antibodies for NPY or non-secretory negative control actin, or no antibody (ø mAb) are placed in the wall of the left ventricle and cycled with the detection/reset protocol described herein. NPY release is evoked either by ectopic pacing of the right ventricle (Right vent. Paging) or bilateral stellate ganglion stimulation (BSG, 10 Hz, 2 times threshold). Specific, time-resolved release of NPY in response to stimuli is provided in FIG. 18 and FIG. 19, demonstrating specificity compared to actin (no-secreted control protein) in a non-saturating, time-resolved manner, and validating a specific, non-saturating, localized, high time resolution measure of protein/neurotransmitter in a living, moving tissue.

In some embodiments, at least one of the electrodes selected from the group consisting of the measurement electrode and the reference electrode are made of platinum for placement in living tissues or vasculature. In some embodiments, at least one electrode is an electrode selected from the group consisting of: wire electrodes, microwire electrodes, needle electrodes, plunge electrodes, penetrating electrodes, patch electrodes, 2D shank electrodes, 3D shank electrodes, and multi-electrode arrays. In some embodiments, the electrode has a conductive substrate layer deposited on the electrode surface suitable for attachment/binding of IgG antibodies, IgG binding fragments (Fab), single-domain antibody fragments, and peptide binding domain fragments. In some embodiments, the conductive substrate layer is polydopamine. Polydopamine is bound to the sensing surface through electrodeposition. Polydopamine presents a highly reactive substrate for covalent binding of the trap molecules as described above. In some embodiments, the biochemical compound is a protein or peptide that specifically binds to the signaling molecule (e.g. a specific antibody/antibody fragment raised against the signaling protein of interest). In some embodiments, the one or more electrodes are placed into the tissue/organ via direct access or via transcutaneous access. In some embodiments, the one or more electrodes are inserted via vascular access, the electrode(s) advanced to the tissue/organ of interest and advanced into that tissue/organ. In some embodiments, the one or more electrodes are inserted via vascular access and advanced to adjacent to or remote vascular sites. In some embodiments, a plurality of electrodes is placed at a plurality of locations within and around the tissue/organ to assess regional differences in the abundance of the biochemical compound.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: Real Time Catecholamine Detection in the Heart

Experiments were conducted to examine whether catecholamines can be detected within the heart using FSCV. A flexible electrode was implanted into the ventricular wall of the beating heart of an anesthetized pig (FIG. 10). The left anterior descending (LAD) artery was occluded above the implanted electrode (FIG. 1, FIG. 12A through FIG. 12C), and norepinephrine was measured by the electrode using FSCV. A kymograph (FIG. 12B) was created depicting oxidation potential plotted over voltage and time. In response to LAD occlusion, an increase in current is observed at the primary oxidation potential that lasts the duration of the occlusion before dissipation. Analysis of voltammograms at defined time points, before and during LAD occlusion, allows for visualization of peak potentials of the oxidation potential. Plotting the primary oxidation potential for norepinephrine as a function of time demonstrates the real-time dynamics of norepinephrine detection during LAD occlusion (FIG. 1 and FIG. 12C).

Experiments were also conducted using multiple electrodes positioned in different regions of the heart to measure norepinephrine in the heart during LAD occlusion. FSCV currents were measured in regions of the heart relative to the induced ischemic zone. FIG. 12B depicts a kymograph from one of the electrodes prior to, during, and following manual arterial occlusion protocol, demonstrating an increased oxidation current characteristic for norepinephrine. FIG. 1 and FIG. 12C depict the data from all 4 channels, demonstrating the ability to measure FSCV at high time resolution in sub regions of the heart.

### Example 2: Peptide Detection

In order to determine whether specific chromaffin granule contents could be detected in intact tissue, carbon fiber electrodes were functionalized by covalently linking anti-enkephalin antibodies to the distal tip. Sample recordings are provided in FIG. 14A through FIG. 16 to demonstrate specificity of the probe for enkephalin versus non-specific Glyceraldehyde 3-phosphate dehydrogenase (GAPDH). Then, paired electrodes were prepared for enkephalin (positive) and a non-secretory negative control peptide, GAPDH (in vitro calibration, FIG. 15A through FIG. 16). Signals for enkephalin (Enk) and GAPDH electrodes were acquired under a time-domain approach, including a two-step depolarization to avoid cross contamination by non-specific amperometric signals, and processed to measure the total charge input (charge (Q)=capacitance (C)*voltage (V)) with a change in current amplitude serving an index of the change in capacitance. Resulting signals were specific for the Enk electrode as expected and a cross-calibration to a standard curve obtained under in vitro conditions revealed a signal indicating 132 picomolar pM Enk release, a value well within that expected and determined by other means (FIG. 16).

### Example 3: Fast In Vivo Detection of Myocardial Norepinephrine Levels in the Beating Porcine Heart

Cardiac sympathetic activation occurs during stress and exercise to improve cardiac output. However, in the setting of cardiac injury, a decrease in cardiac output reflexively results in chronic sympathetic activation, which can lead to progression of heart failure and development of ventricular arrhythmias (Fukuda K et al., Circulation research. 2015 Jun. 5; 116(12):2005-19). Norepinephrine (NE) is the primary neurotransmitter released from post-ganglionic sympathetic efferents (Janig W. Functional anatomy of the peripheral sympathetic and parasympathetic system. The integrative action of the autonomic nervous system: neurobiology of homeostasis. 2006:13-34). Heart failure is known to result in elevated myocardial NE levels which portend a worse prognosis and are associated with cardiac mortality, ventricular arrhythmias, and sudden cardiac death (Cohn J N et al., New England journal of medicine. 1984 Sep. 27; 311(13):819-23). Therefore, NE can serve an important biomarker of the status of cardiac disease. However, current methods to detect NE have significant limitations, and as a result, measurements of NE levels have not been routinely used clinically to assess the status of cardiac disease and to adjust therapies. In particular, measure of myocardial NE relies on lengthy collections of interstitial NE in cardiac tissue through deployment of microdialysis tubes passing through the myocardium, which in addition to requiring large volumes, sample preparation and handling, has a subsequent delay in analysis. Cardiac imaging modalities such as positron emission tomography (PET) (Fallavollita J A et al., Journal of the American College of Cardiology. 2014 Jan. 21; 63(2):141-9) and metaiodobenzylguanidine (MIBG) (Dae M W, Journal of thoracic imaging. 1990 July; 5(3):31-6) have therefore been developed to assess sympathetic innervation. However, these modalities provide a one-time static measurement, are costly, and suffer from poor resolution. Thus, while having the potential to provide important diagnostic and prognostic information on the status of autonomic control of the heart, traditional approaches to NE measurement have been limited in key aspects of temporal resolution, sample preparation, resolution of variation of response and time for signal processing.

In the present study, a novel adaptation of a dynamic approach for electrochemical detection of NE levels in vivo is presented. The approach is based on Fast Scanning Cyclic Voltammetry (FSCV), a method utilized to measure catecholamine release from isolated cells (Leszczyszyn D J et al., Journal of neurochemistry. 1991 June; 56(6):1855-63; Pihel K et al., Analytical Chemistry. 1994 Dec. 1; 66(24):4532-7) or from tissues (Jaffe E H et al., Journal of Neuroscience. 1998 May 15; 18(10):3548-53; Walsh P L et al., American Journal of Physiology-Cell Physiology. 2011 January; 300(1):C49-57; Wolfe J T et al., The Journal of physiology. 2002 January; 538(2):343-55). Briefly, an electrode is placed near the source of the transmitter and its potential driven though the oxidation/reduction potentials by a voltage-clamp circuit. Thus, as the electrode potential is driven positive to the oxidation potential for NE, the NE is oxidized to a quinone product. The oxidation reaction generates electrons that are then measured as a compensating current in the voltage clamp and report the detection of molecules of NE. Driving the electrode potential back to a negative polarization reduces the quinone product to regenerate the catecholamine (Chow R H, and von Ruden L. Chapter 11. Electrochemical detection of secretion from single cells. In: Single-Channel Recording, Second Edition, edited by Sakmann B, and Neher E. New York: Plenum Press, 1995, p. 245-275). Traditionally for these applications, electrodes for NE measurement were made of small diameter carbon fibers encased in a pulled borosilicate glass capillary or polypropylene tube to stabilize and insulate the brittle carbon fiber electrode and electrode placement was with the aid of a micromanipulator. While this configuration is very effective at measuring voltammetric currents in isolated cell or tissue applications, it suffers from several limitations that make measurements in a large, moving preparation impossible (e.g. probe length and flexibility, head stage design, proximity requirement, reference electrode placement). The primary objective of this study was to evolve an FSCV technology that circumvents these limitations and is capable of recording local interstitial NE at high temporal resolution from multiple regions of the beating heart.

The materials and methods are now described.

Instrumentation

A multichannel amplifier was designed that incorporated a low-resistance feedback resistor in the voltage-clamp circuit in order to charge the greater capacitance of the long, flexible electrode, while still supporting a sufficient dV/dt scan rate. The custom amplifier design was based on the NPI VA-10M, multichannel amplifier (NPI Electronic, Tamm, Germany). A 3-electrode design was employed to accommodate placement of sensing electrodes in the myocardium and reference/ground electrodes in the chest wall. The amplifier was fitted with a 5× command potential input to allow scans up to 1.2 V to allow measure of epinephrine and for specific isolation of NE over other catecholamines (Wolf K et al., Physiological reports. 2016 September; 4(17).e12898). The command potential was issued through software via the digital-to-analog converter channels, and signal acquired through the analog-to-digital converter channels of a HEKA LIH 8+8 analog-to-digital/digital-to-analog device (HEKA Elektonic, Holliston, Mass.). Other unique features of the amplifier included a switchable feedback resistor for each of the 4 acquisition channels, allowing for the choice of 1 MOhm or 10 MOhm feedback circuit to accommodate electrode variability on a single channel basis. A single head stage with a common ground/reference circuit for all 4 acquisition channels was also developed in order to place the device near the chest in a single physical unit. All data reported here were collected with the 1 MOhm feedback resistor setting.

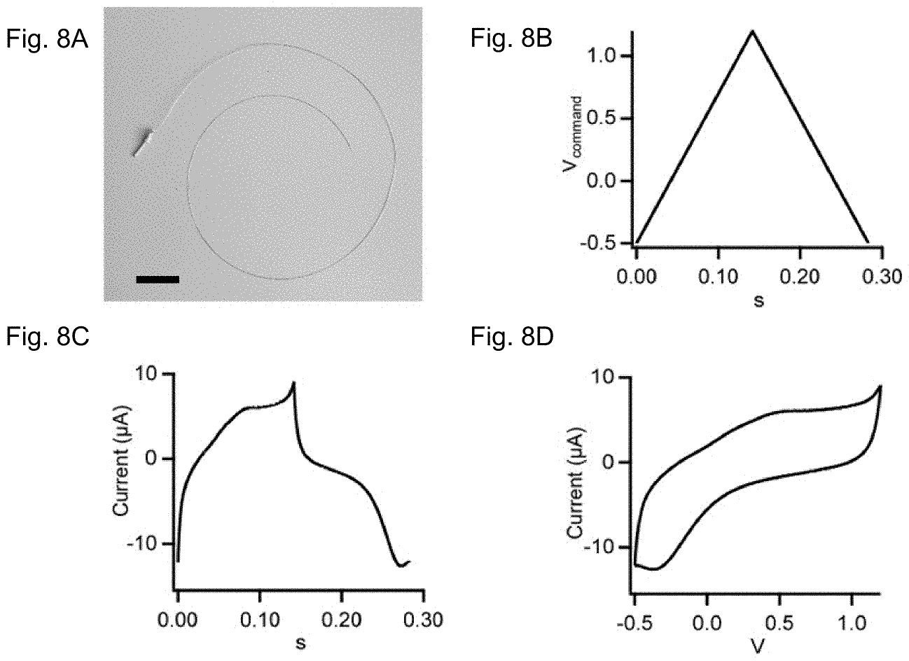

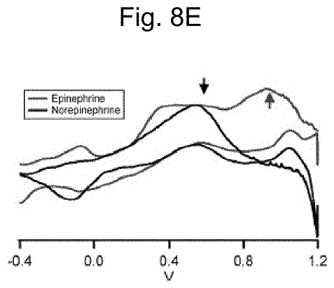

Platinum (Pt) wire electrodes, 30 cm in length and 127 μm in diameter (PFA137 coated, A-M Systems, Sequim, Wash.)), served as sensing elements for in vivo FSCV (FIG. 8A through FIG. 8D and FIG. 11A through FIG. 11D). On one end, the PFA coating was stripped to reveal approximately 5 mm bare wire that was then crimped into a 1 mm gold plated connector pin. The wire-pin joint was stabilized by flowing a small amount of solder into the joint (FIG. 8A). Admittance analysis was performed on multiple Pt electrodes, and it was found that the phase offset for these electrodes, at the scan rate utilized to collect data in this report, varied electrode to electrode, but was between 33 and 51 degrees. This phase offset was accounted for in analysis of voltammograms and oxidation currents.

Acquisition and Analysis Software

Software for driving command potential and data acquisition was custom written in IGOR Pro (v. 7.08 WaveMetrics, Lake Oswego, Oreg.). The LIH 8+8 issued command voltage and acquired data from the custom NPI VA-10M amplifier. Filter and gain were telegraphed from the amplifier. These values and recording parameters were written into the headers of the data waves for record keeping. Data were filtered at 1 kHz through a 2-pole analog Bessel filter and digitized at 10 kHz. The command potential for FSCV was a sawtooth waveform between −0.5 V and 1.2 V, issued at 12 V·s⁻¹, for an effective cycle rate of approximately 3.5 Hz. Collected data were baseline subtracted by an average voltammogram composed of 10 cycles prior to the experimental perturbation. Data for each channel were converted into a kymograph with command voltage plotted against time, each column representing a single scan (FIG. 12A through FIG. 12C). Current amplitude was indicated by color. A horizontal line profile, representing current amplitude at a given command potential, was extracted at the oxidation potential for norepinephrine, corrected for the phase offset due introduced by the electrode capacitance. An additional initial current artifact, due to equilibration of the electrode redox status under the sawtooth command potential, was subtracted for presentation. Data were saved in a 3-dimensional pooled data wave for further statistical analysis and archive.

In Vitro Measurements

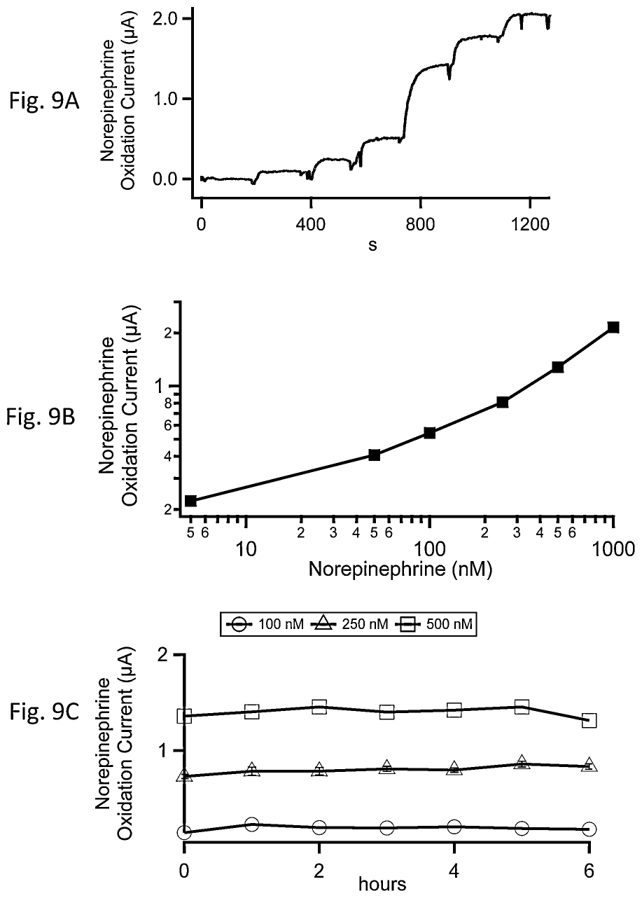

For in vitro measurements (FIG. 5A through FIG. 9C), electrodes were held by a coarse manual manipulator and their tips placed in a laminar flow superfusion chamber (with 2.5 to 3 ml in total fluid volume). Electrodes were superfused at a constant rate of approximately 2 ml·minute⁻¹ with bicarbonate-buffered saline (BBS) of the following composition (in mM): 140 NaCl, 26 NaHCO₃, 3.5 Glucose, 3 CaCl₂, 2 KCl, 2 MgCl₂. Calcium chloride was added from stock solution (3 M) prior to recording to avoid precipitation as CaCO₃. The saline were constantly bubbled with 5% CO₂ and 95% O₂ to maintain the pH level around 7.4. Variable concentrations of NE in BBS were sequentially perfused into the chamber with concentrations ranging between 0 and 2 μM. Oxidation currents were determined for each level of NE. Stability of recording over 6 h was assessed by repeating measuring a constant given level of NE (100, 250 and 500 μM) in BBS (FIG. 9C).

In Vivo Measurements

All animal experiments were approved by the University of California-Los Angeles Animal Research Committee and performed in accordance with guidelines set forth by the National Institutes of Health Guide for the Care and Use of Laboratory Animals (8th edition, 2011). Adult Yorkshire pigs, n=4 (2 males and 2 females), were sedated with intramuscular telazol (4-6 mg/kg), intubated, and mechanically ventilated. General anesthesia was maintained with inhaled isoflurane (1.5-2.5%) and intravenous boluses of fentanyl (total: 10-30 μg/kg) during surgical preparation. Continuous intravenous saline was infused through the femoral vein throughout the experiments to maintain volume homeostasis. Arterial blood pressure was measured via a femoral arterial line. Heart rate was monitored by lead II ECG. Left ventricular (LV) systolic pressure was measured using a pressure monitoring pigtail catheter (5 Fr) inserted into the LV via the left carotid artery and connected to a PCU-2000 pressure control system (Millar Instruments, Houston, Tex.). Arterial blood gas was tested hourly and adjustment of ventilation and/or administration of sodium bicarbonate were made as necessary to maintain acid-base homeostasis.

A median sternotomy was performed to expose the heart, as well as the stellate ganglia, inferior vena cava (IVC), and descending thoracic aorta. Snare occluders were placed around the great vessels (inferior vena cava, IVC, and descending aorta) and at the first diagonal branch of the left anterior descending coronary artery (LAD). The stellate ganglia were isolated behind the parietal pleura, bipolar electrodes were placed into each stellate ganglion, and connected to a stimulator with an isolation unit (Grass Technologies, S88 and PSIU6, Warwick, R.I.). For each stellate ganglion, cardiac-related threshold was defined as the current that evoked a 10% increase in heart rate or systolic blood pressure at 4 Hz frequency and 4 ms pulse width. A bipolar cardiac pacing catheter was inserted into the right ventricle via the right jugular vein and connected to a Micropace system ((EPS320; Micropace, Canterbury, New South Wales, Australia) for ventricular pacing. Following the completion of surgery, general anesthesia was changed to α-chloralose (50 mg/kg I.V. bolus with 10 mg/kg/h continuous i.v. infusion).

For insertion into the wall of the heart, FSCV Pt insulated wire electrodes were threaded through a 25-gauge hypodermic needle. The tip of the electrode was pushed to protrude approximately 0.5 mm beyond the needle tip, and was bent back along the shank of the needle to create a barb akin to a fish hook (FIG. 11A). The needle was then inserted into the mid-myocardium of the ventricular wall and the needle withdrawn, leaving the electrode inserted in the ventricle wall. For the purposes of regional analyses, anterior refers to ventral and posterior refers to dorsal aspect of the animal. Electrodes were placed at four sites covering the basal, apical, anterior, and lateral parts of the left ventricle (LV). This configuration produced minimal damage of the heart wall and resulted in stable electrode placement for the duration of the experimental protocol, often lasting 6 hours. Ground and reference electrodes (two 18 gauge syringe needles) were inserted in the chest wall, in intercostal muscle tissue. Following deployment of the multiple FSCV probes, they were cycled for 20 min prior to experimental procedures to establish a stable baseline. Hemodynamics and interstitial NE responses were then measured concurrently at baseline, in response to a given intervention, and then into the recovery phase.

Cardiac Stressors (FIG. 11A Through FIG. 13C)

The transient cardiac stressors tested included: bilateral stellate ganglia stimulation for 4 minutes (4 Hz, 4 ms pulse width, 2× threshold, first 2 min and increasing to 10 Hz for last two min), inferior vena cava occlusion (decrease preload) for 60 seconds, descending aorta occlusion (increase afterload) for 60 seconds, occlusion of the left anterior descending coronary artery for one minute, and intermittent ventricular stimulation to induce variably coupled premature ventricular contractions (PVC) at every 8 heart beats for 60 seconds and then every 4 heart beats for 60 seconds. A minimum of 15 minutes was allowed between stressors for recovery of cardiac function to baseline.

Electrocardiogram (ECG), hemodynamic data, and stimulus markers (reflecting intervention onsets and offsets) were input to a data acquisition system (Cambridge Electronic Design—CED, Power1401, Cambridge, UK). Data were analyzed offline using the software Spike2 (Cambridge Electronic Design). Data streams from the voltammetry and CED data acquisition systems were manually time-synchronized at the time of data collection and merged during subsequent off-line analysis. At the completion of the experiments, animals were euthanized under anesthesia by inducing ventricular fibrillation via application of direct current to the heart.

The results are now described.

Electrode Design and Characterization

Acquisition and analysis software was developed in-house to drive a custom designed 4 channel voltage-clamp amplifier. PFA-insulated platinum wires, 127 μM in diameter and 30 cm in length, were used as flexible FSCV electrodes (FIG. 8A). A sawtooth command waveform (FIG. 8B) drove the recorded voltammograms (FIG. 8C, FIG. 8D). Recordings were performed in bicarbonate-buffered saline (BBS) to mimic the interstitial conditions of the myocardium. A sample voltammogram of an electrode in BBS displays a hysteresis at a scan rate of 12 V/s from −0.5 V to 1.2 V (FIG. 8D). This command potential range is wide enough to measure norepinephrine (NE) as well as other potential catecholamines (e.g. epinephrine) and the scan rate provides a sample rate of approximately 3.53 Hz.

In Vitro Assessments of Electrode Sensitivity and Stability

Electrodes were superfused with BBS supplemented with increasing concentrations of NE (0 to 2 μM) in a laminar flow chamber. Peak currents at the NE oxidation potential were measured and plotted (FIG. 9A). Maximum measured current at the NE oxidation potential is plotted against NE concentration and provides a standard calibration curve (FIG. 9B). In order to account for non-linearity of the standard curve, acquired data are matched point-for-point to their intersection with the standard curve. The result reports a change in NE concentration from baseline. Next, the stability of the recording configuration was tested by recording peak currents at the NE oxidation potential by repeating addition of the given concentrations of NE over 6-hours and found the electrodes to be stable over this period (FIG. 9C) where no significant degradation in measure signal for all three NE levels tested (100, 250 and 500 μM).

In Vivo Assessments of Electrode Sensitivity and Stability

A platinum electrode was inserted into the left ventricle (LV) mid-myocardium with aid of a hypodermic needle (FIG. 10 and FIG. 11A). Interstitial NE levels were evaluated at baseline and in response to bilateral stellate ganglion stimulation. Data are presented as a kymograph (FIG. 11B) with Y-axis columns representing the up-stroke of the sawtooth command potential, and time represented on the X axis. Current magnitude is color-coded. The black horizontal line represents the peak oxidation potential for NE. There is emergence of a signal during stellate ganglia stimulation, which persists somewhat after stimulation, indicating increased NE at the electrode tip. Example voltammograms (current vs. command potential) are provided in FIG. 11C. The black voltammogram was measured at baseline (time-point indicated by the black arrow in FIG. 11B), and the blue during stellate ganglia stimulation (time-point indicated by the blue arrow in FIG. 11B). Currents were pulled from the kymograph, as a function of time, at the peak NE oxidation potential (black line in FIG. 11B) and calibrated against the standard curve to provide time-resolved, evoked changes in NE concentration (FIG. 11(C, bottom). These data show a significant increase in NE evoked by stellate stimulation. This approximate 600 nM increase in NE is quite consistent with values obtained through other techniques (i.e. radio immune-assay (Killingsworth C R et al., Circulation. 2004 May 25; 109(20):2469-74; Tallaj J et al., Circulation. 2003 Jul. 15; 108(2):225-30)). In simultaneous hemodynamic measurements, complementary increases in heart rate (HR), LV peak systolic pressure (LVSP) and LV developed pressure (dP/dt) were recorded during stellate ganglia stimulation (FIG. 11D).

A major goal of our study was to measure interstitial NE levels across multiple regions of the myocardium. Next, experiments were conducted utilizing 4 independent acquisition channels to provide a gross spatial map of NE levels across the left ventricle in response to acute occlusion of the left anterior descending coronary artery (LAD, 180 s duration). LAD occlusion results in loss of circulation beyond the occlusion site, and subsequent regional ischemia in the ventricular apex. Subsequent activation of local nociceptors produces a reflex sympatho-excitation (Foreman R D et al., Comprehensive Physiology 5: 929-960, 2015; Longhurst J C et al., Annals of the New York Academy of Sciences. 2001 June; 940(1):74-95; Malliani A et al., Brain Research. 1975 April; 87(2-3):239-246) which results in release of NE. Electrodes were placed caudal to the site of vessel occlusion (indicated by black arrow) within basal regions of the LV whose circulation remains intact (indicated by green and black dots, FIG. 12A). Another set of electrodes were placed apical to the site of occlusion where circulation is blocked (indicated by red and blue dots). FSCV was performed spanning a time-frame 60 s prior to, during occlusion, and into the reperfusion phase. FIG. 12B provides the kymographs for each channel (indicated by the colored dot to the left of each kymograph). As in FIG. 11B, black horizontal lines indicate the peak potential for NE oxidation. Line profiles for current magnitude were pulled as a function of time from the kymographs, calibrated against the standard curve, and plotted (FIG. 12C). These data demonstrate that myocardium apical to the occlusion site (red, blue dots) exhibited a strong elevation in interstitial NE while those regions of the left ventricle receiving normal circulation (green and black dots) did not demonstrate an increase in NE levels beyond baseline. Thus, this approach is capable of providing spatially-resolved, high temporal resolution readouts of local NE release under cardiac ischemia and stress.

Lastly, NE measurements under varied autonomic and cardiac interventions were correlated to hemodynamic responses measured simultaneously in the same test preparation. Four electrodes were placed across the left ventricle, one basal, one apical, and two lateral. NE release was evaluated during transient occlusions of the descending aorta (AO; FIG. 13A; an increase in afterload) or inferior vena cava (IVC; FIG. 13B: a decrease in preload) and induction of premature ventricular contractions via programmed pacing (PVC; FIG. 13C). As expected, the aortic occlusion resulted in decreased interstitial NE levels, followed by a rebound after release of the occlusion. Additionally, both inferior vena cava occlusion and ectopic stimulation increased interstitial NE as expected. Hemodynamic parameters (LVSP, HR and dP/dt) mirrored the evoked changes in NE concentration and are presented in the right column. Thus, NE measured by FSCV, in the beating heart, correlates with well-characterized physiological responses to autonomic stressors.

Electrochemical approaches for catecholamine detection have been well established in the fields of neuroscience and analytic chemistry. Steady state (i.e. fixed potential) amperometric detection of catecholamine release from isolated neuroendocrine chromaffin cells represented a breakthrough in the study of the molecular basis of neurotransmitter exocytosis (Chow R H et al., Nature. 1992 March; 356(6364):60-3; Jankowski J A et al., Journal of Biological Chemistry. 1992 Sep. 15; 267(26):18329-35). Indeed, this implementation of electrochemical detection exhibits sub millisecond resolution, that it is has been a key tool in the study of fusion pore regulation in the secretion process, able to measure the rate of release of catecholamine through single fusion pores (Fulop T et al., Archives of biochemistry and biophysics. 2008 Sep. 1; 477(1):146-54; Wang C T et al., The Journal of physiology. 2006 January; 570(2):295-307). However, steady state amperometry suffers from the limitation that it cannot determine which type of oxidizable substance is being released (Chow R H, and von Ruden L. Chapter 11. Electrochemical detection of secretion from single cells. In: Single-Channel Recording, Second Edition, edited by Sakmann B, and Neher E. New York: Plenum Press, 1995, p. 245-275), thus it is not appropriate for tissue-level studies where multiple oxidizable molecules may be present.

Fast scanning cyclic voltammetry (FSCV) relies on scanning the probe potential through the range of oxidation potentials of many substances. Identification of which substance is oxidizing is accomplished through measuring the specific oxidation potential (i.e. separating norepinephrine from dopamine) or by measuring the full spectrum of oxidation reactions (i.e. norepinephrine from epinephrine). In this dynamic electrochemical approach, the electrode potential is driven by a voltage clamp circuit with a dynamic command potential spanning the oxidation-reduction potentials for NE. Thus, as the electrode potential is driven in a positive dV·dt⁻¹ past the oxidation potential for NE, the NE is oxidized to a quinone product and releasing 2 electrons. These electrons are then measured as a compensating current in the voltage clamp and report the detection of a single molecule of NE (Pihel K et al., Analytical Chemistry. 1994 Dec. 1; 66(24):4532-7). Driving the electrode potential back to a negative polarization reduces the quinone product to regenerate the catecholamine. Here, a form of FSCV appropriate to measure norepinephrine was devised at discrete locations in the myocardium with minimal tissue damage, fast sample frequency, rapid data analysis and in multiple parallel channels. Long (30 cm), flexible platinum PFA-insulated electrodes were developed and characterized to reach the heart in an open chest porcine model. Additionally, the circuitry design of a commercially available, multi-channel voltammetry amplifier was revised to meet the accommodate capacitance of the platinum electrodes and to provide a stable and accurate reference potential for the voltage clamp circuitry.

Platinum electrodes are very commonly used in nerve recordings. They are flexible, available in a variety of diameters, provide a low level of reactivity and do not readily corrode. Thus, they exhibit several characteristics required to be used on the dynamic context of open-chest heart recordings. One of the proprieties of platinum is that they are not a purely capacitive material, meaning that when a voltage is applied, they do transfer charge into the surrounding tissue. This characteristic defines a limitation on the electronics used to clamp the electrodes to the desired command potential. One must be able to push significant current to charge the capacitance of the electrode to clamp it to the command potential. As described above, a custom device was developed for this purpose. This amplifier incorporates 4 individual and separately-controlled voltage-clamp channels with switchable gain, filter and command potential inputs. The single head stage connects to and drives 4 independent electrodes but utilizes a single reference/ground circuit for all 4 channels. The head stage was designed to be switchable between a 1 and 10 MOhm feedback resistor, which is low enough to push significant current required and high enough to provide a reliable voltage clamp of the electrode while providing a large range.

Neural control of the heart reflects a hierarchy of interdependent reflex loops involving intrathoracic and central nervous system neural networks (Ardell J L et al., Comprehensive Physiology. 2011 Jan. 17; 6(4):1635-53). The efferent outputs for the cardiac nervous system are the parasympathetic and sympathetic neurons (Janig W. Integrative action of the autonomic nervous system: Neurobiology of homeostasis. Cambridge University Press; 2008 Jun. 26; Levy M N, and Martin P J. Neural control of the heart. In: Handbook of Physiology: Section 2: The Cardiovascular System, Volume 1: The Heart, edited by Berne R M. Bethesda: The American Physiological Society, 1979, p. 581-620). At rest, there is a parasympathetic predominance that shifts to a sympathetic dominance during high levels of stress (Ardell J L et al., The Journal of Physiology. 2016 Jul. 15; 594(14):3877-909; Levy M N, and Martin P J. Neural control of the heart. In: Handbook of Physiology: Section 2: The Cardiovascular System, Volume 1: The Heart, edited by Berne R M. Bethesda: The American Physiological Society, 1979, p. 581-620). Cardiac disease disrupts not only heart muscle, but also the cardiac nervous system (Ajijola O A et al., JCI insight. 2017 Sep. 21; 2(18); Rajendran P S et al., The Journal of Physiology. 2016 Jan. 15; 594(2):321-41; Vaseghi M et al., JCI insight. 2017 Aug. 17; 2(16)). Both the progression of heart failure and the potential for sudden cardiac death are associated with excessive sympatho-cardiac excitation (Florea V G et al., Circulation Research. 2014 May 23; 114(11):1815-26; Fukuda K et al., Circulation Research. 2015 Jun. 5; 116(12):2005-19). Heterogeneous and high levels of sympathetic output to the heart are major risk factors for morbidity and mortality (Florea V G et al., Circulation Research. 2014 May 23:114(11):1815-26; Fukuda K et al., Circulation Research. 2015 Jun. 5; 116(12):2005-19; Hanna P et al., Cardiac Failure Review. 2018 August; 4(2):92). While direct nerve recordings of sympathetic firing provides an index of neuronal activity (Hart E C et al., American Journal of Physiology-Heart and Circulatory Physiology. 2017 May 1:312(5):H1031-51), measurement of catecholamine levels directly within the heart would provide the most relevant measure of neurotransmitter-receptor interactions, especially when evaluating autonomic tone or assessing regional NE release. This has a high degree of relevance, especially in structural heart disease where heterogeneities in the cardiac electrical substrate are amplified by disparate levels of NE leading to high risk for ventricular arrhythmias including tachycardia/fibrillation (Fukuda K et al., Circulation Research. 2015 Jun. 5; 116(12):2005-19) and where an increased cardiac sympathetic tone as indicated by increased NE levels portends a poor prognosis (Cohn J N et al., New England Journal of Medicine. 1984 Sep. 27; 311(13):819-23). Current approaches for functional readouts of cardiac NE using microdialysis cannot be easily performed in humans and are severely limited in their spatial/temporal readout capability and most often provide data only after significant time delay. Cardiac sympathetic imaging suffers from poor regional resolution in addition to significant time delay and is costly.

Using the approach and multiple interfaces dispersed throughout the left ventricle, it was demonstrated that it is possible to obtain high-resolution dynamic readouts of catecholamine interstitial levels at baseline and in response to stress with FSCV. Proof of concept for this approach is shown in response to direct electrical stimulation of the sympathetic post-ganglionic projections to the heart, transient myocardial ischemia, changes in preload and afterload, and in response to induced premature ventricular contractions, all interventions that can alter sympathetic output to the heart. As expected, some of interventions evoked similar changes in NE throughout the ventricles (e.g. stellate ganglia stimulation and PVC's). Other stressors, especially regional myocardial ischemia, caused disparate release of NE. Importantly, it was shown that these regional NE readouts as provided by FSCV, are stable over time and have a dynamic range that covers the NE concentrations up to and including pathological levels (Arora R C et al., American Journal of Physiology-Regulatory, Integrative and Comparative Physiology. 2003 November; 285(5):R1212-23). When paired with high-density recording of regional cardiac electrical/mechanical function, this technology holds great promise in unraveling the mechanisms underlying arrhythmia formation and pump failure.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for detecting a biochemical compound comprising the steps of:
inserting one or more electrodes in one or more locations selected from the group consisting of: a tissue, an organ, a neural structure, a lymphatic vessel, a lymphatic node, an extravascular fluid compartment, and a peripheral blood vessel;
applying a voltage scan to the electrode; and
detecting a current indicative of the presence and abundance of the compound.

2. The method of claim 1, wherein the one or more electrodes are placed into the myocardium of a heart.

3. The method of claim 1, wherein the one or more electrodes are inserted via epicardial or vascular access.

4. The method of claim 1, wherein the compound is at least one catecholamine selected from the group consisting of norepinephrine and epinephrine.

5. The method of claim 1, wherein at least one electrode is an electrode selected from the group consisting of: wire electrodes, microwire electrodes, needle electrodes, plunge electrodes, penetrating electrodes, patch electrodes, single shank electrodes, 2D shank electrodes, 3D shank electrodes, and multi-electrode arrays.

6. The method of claim 1, wherein the voltage scan is a fast scanning cyclic voltammetry (FSCV) voltage scan.

7. The method of claim 6, wherein the FSCV voltage scan comprises a waveform selected from the group consisting of: a sawtooth pattern and sinusoidal pattern.

8. The method of claim 1, wherein the method comprises detecting the oxidation current of the compound.

9. The method of claim 1, wherein the method comprises constructing a voltammogram from the detected current, thereby identifying the compound.

10. The method of claim 9, comprising quantifying the abundance of the compound by plotting the peak current on a calibration curve.

11. The method of claim 1, wherein the organ is a heart, and the one or more electrodes are placed in one or more locations selected from the group consisting of: a coronary sinus of the heart, a great vein of the heart, vena cava, left ventricle, aorta, right ventricle, right atria, left atria, pulmonary veins, pulmonary artery, stellate ganglia, dorsal root ganglia, epicardial fat pad, and pericardial fat pad.

12. The method of claim 1, wherein the presence and abundance of the biochemical compound is assessed in response to one or more cardiac stressors.

13. The method of claim 1, wherein a plurality of electrodes are placed at a plurality of locations within and around a heart to assess regional differences in the abundance of the biochemical compound.

14. A method for detecting a biochemical compound comprising the steps of:
inserting one or more electrodes in one or more locations selected from the group consisting of: a tissue, an organ, a neural structure, a lymphatic vessel, a lymphatic node, an extravascular fluid compartment, and a peripheral blood vessel, wherein at least one electrode comprises a receptor molecule that specifically binds the biochemical compound; and
detecting a change in the capacitance of the electrode thereby indicating the presence of the biochemical compound.

15. The method of claim 14, wherein the biochemical compound is a protein or peptide that specifically binds to the receptor molecule.

16. The method of claim 14, wherein the level of the compound is detected in at least one ganglia selected from the group consisting of intrathoracic ganglia, stellate ganglia, autonomic ganglia, nodose ganglia, dorsal root ganglia and petrosal ganglia.

17. The method of claim 14, wherein the one or more electrodes are placed in a peripheral artery or peripheral vein.

18. The method of claim 14, wherein the one or more electrodes are placed into a tissue or organ via direct access.

19. The method of claim 14, wherein the one or more electrodes are placed into a tissue or organ via transcutaneous access.

20. The method of claim 14, wherein the one or more electrodes are placed into a tissue or organ via vascular access.

21. A biochemical compound detection device, comprising:
a controller, comprising a voltage clamp circuit and signal acquisition and amplification device;
a reference electrode communicatively connected to the controller; and
a one or more measurement electrodes communicatively connected to the controller;
wherein the controller is configured to measure a reference potential across the reference and ground electrodes and voltage clamp of the one or more measurement electrodes relative to the reference potential with a defined sawtooth, sinusoidal or step command potential, and to measure the current passing through the one or more measurement electrodes over time; and
wherein the measurement electrodes are configured to measure the presence and concentration of one or more biochemical compounds.

22. The biochemical compound detection device of claim 21, further comprising a ground electrode, wherein the controller is configured to measure an electric potential between the reference electrode and the ground electrode.

23. The biochemical compound detection device of claim 21, wherein at least one measurement electrode comprises a receptor molecule that specifically binds to a biochemical compound.

24. The biochemical compound detection device of claim 23, further comprising a semi-permeable membrane applied to a portion of an electrode selected from the group consisting of the reference electrode, the measurement electrode, and the ground electrode.

25. The biochemical compound detection device of claim 21, wherein at least one of the electrodes selected from the group consisting of the measurement electrode and the reference electrode are made of platinum.

26. The biochemical compound detection device of claim 21, wherein the reference electrode and one or more measurement electrodes are selected from the group consisting of: wire electrodes, microwire electrodes, needle electrodes, plunge electrodes, penetrating electrodes, patch electrodes, single shank electrodes, 2D shank electrodes, 3D shank electrodes, and multi-electrode arrays.

27. The biochemical compound detection device of claim 21, wherein the reference electrode and one or more measurement electrodes each has a conductive substrate layer deposited on the electrode surface suitable for attachment/binding of IgG antibodies, IgG binding fragments (Fab), single-domain antibody fragments, and peptide binding domain fragments.

28. The biochemical compound detection device of claim 27, wherein the conductive substrate layer is polydopamine.

29. The biochemical compound detection device of claim 21, wherein the controller further comprises a voltage clamp, configured to maintain a substantially constant voltage across two or more electrodes.

30. A biochemical compound detection device, comprising:
a controller, comprising a voltage clamp amplifier;
a reference electrode communicatively connected to the controller;
a ground electrode communicatively connected to the controller; and
one or more sensing electrodes communicatively connected to the controller, each of the one or more sensing electrodes being voltage clamped to a template of positive and negative voltage steps;
wherein the controller is configured to measure an electric potential across the reference electrode, the ground electrode, and to apply a command potential relative to the reference potential through a voltage clamp to one or more sensing electrodes, and to measure the current passing through one or more sensing electrodes over time; and
wherein one or more sensing electrodes are configured to measure the presence and concentration of one or more biochemical compounds.

31. The biochemical compound detection device of claim 30, wherein sensitivity of the device is reset by applying a negative potential pulse configured to expel target molecules from capture agents on each of the one or more sensing electrodes, readying the capture agents for a subsequent binding of target molecules for further detection events.
